(19) United States
(12) Patent Application Publication
(10) Pub. No.: US 2023/0417752 A1
(43) Pub. Date: Dec. 28, 2023

(54) IMMUNOHISTOCHEMISTRY (IHC) PROTOCOLS AND METHODS FOR DIAGNOSING AND TREATING CANCER

(71) Applicant: Agilent Technologies, Inc., Santa Clara, CA (US)

(72) Inventors: Sara ALVAREZ, Carpinteria, CA (US); Quyen NGUYEN, Denver, CO (US); Nichole LAPOINTE, Santa Barbara, CA (US); Michael DIMAIO, Loveland, CO (US); Mariana CAJAIBA, Loveland, CO (US); Russell A BALDOCCHI, Loveland, CO (US)

(21) Appl. No.: 18/208,178

(22) Filed: Jun. 9, 2023

Related U.S. Application Data
(60) Provisional application No. 63/356,000, filed on Jun. 27, 2022.

(51) Int. Cl. G01N 33/574; G01N 33/50

(52) U.S. Cl. CPC G01N 33/574; G01N 33/5011

(57) ABSTRACT

In alternative embodiments, provided are immunohistochemistry (IHC) methods for determining and scoring reproducibly the extent of expression of the protein carcinoembryonic antigen-related cell adhesion molecule 5, or CEACAM5, in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring CEACAM5 expression and utilizing the score as a companion or complementary diagnostic or to treat or ameliorate cancer or a tumor.

### TECHNICAL FIELD

This invention generally relates to cancer treatments, companion or complementary diagnostics and immunohistochemical methods. In alternative embodiments, provided are immunohistochemistry (IHC) methods for determining and scoring reproducibly the extent of expression of the protein carcinoembryonic antigen-related cell adhesion molecule 5, or CEACAM5 (also known as CD66e (Cluster of Differentiation 66e)), in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring CEACAM5 expression and utilizing the score as a companion or complementary diagnostic or to treat or ameliorate cancer or a tumor.

### BACKGROUND

Carcinoembryonic antigen-related cell adhesion molecule 5 (CEACAM5), also known as CD66e (Cluster of Differentiation 66e), is a member of the carcinoembryonic antigen (CEA) gene family. There is evidence that high CEACAM5 expression is firmly associated with CD133-positive colorectal cancer stem cells. CEACAM5 is used as a clinical biomarker for gastrointestinal cancers and may promote tumor development through its role as a cell adhesion molecule. Additionally, CEACAM5 may regulate differentiation, apoptosis, and cell polarity.

Carcinoembryonic antigen-related cell adhesion molecule 5 (CEACAM5) is a cell-surface glycoprotein overexpressed in several tumor types, including those of the gastrointestinal tract, genitourinary system, breast cancer, and non-squamous NSCLC. CEACAM5 is a member of the CEA family of proteins that plays a key role in cell migration, cell invasion, and cell adhesion.

### SUMMARY

In alternative embodiments, provided are immunohistochemistry (IHC) methods for determining and scoring the extent of cellular membrane expression of carcinoembryonic antigen-related cell adhesion molecule 5, or CEACAM5 (also known as CD66e (Cluster of Differentiation 66e)) in a tissue sample, comprising:
(a) staining a tissue sample with an antibody which specifically binds to CEACAM5;
(b) determining a total number of viable tumor or cancer cells having CEACAM5 cellular membrane staining, and determining a total number of staining and non-staining viable tumor or cancer cells in at least a portion of the tissue sample,
   wherein a tumor or cancer cell is counted as positively stained with anti-CEACAM5 antibody if there is CEACAM5 cellular membrane staining at any intensity above a defined threshold; and
(c) determining a Tumor Intensity Proportion Score (TIPS), wherein the Tumor Intensity Proportion Score is the number of CEACAM5 staining viable tumor or cancer cells found in the tissue sample divided by the total number of staining and non-staining viable tumor or cancer cells, multiplied by 100.

In alternative embodiments of IHC methods as provided herein:
the CEACAM5 cellular membrane staining comprises whole membrane staining, discontinuous membrane staining, partial membrane staining, complete membrane staining, punctate membrane staining, linear membrane staining, luminal membrane staining, apical membrane staining, basal membrane staining, lateral membrane staining, basolateral membrane staining, high cytoplasmic staining at a 2+ or greater positive staining intensity, high cytoplasmic staining at a 3+ positive staining intensity, or a combination thereof;
the total number of viable tumor or cancer cells comprises luminal cells, glandular luminal cells, intraluminal cells, multiple layers of cells, cells having signet ring morphology, high cytoplasmic staining cells, cells with intracytoplasmic lumina, or a combination thereof;
the total number of viable tumor or cancer cells counted as positively stained comprises all glandular luminal cells having a cellular membrane associated with the positively staining lumen;
the tissue sample comprises at least about 100 cells;
a tumor or cancer cell counted as positively stained excludes tumor or cancer cells having cytoplasmic staining, staining of normal or non-neoplastic structures, staining nonviable tumor or cancer cells, necrotic cells, cellular debris, stromal staining, or edge artifact staining on a periphery of the tissue sample;
cytoplasmic staining comprises cytoplasmic staining at a 2+ or lesser positive staining intensity; or, the defined threshold comprises a 2+ or greater positive staining intensity; or, the defined threshold comprises a 2+ positive or a 3+ positive staining intensity evaluated at a magnification of from about 4× to about 10×; or further comprising confirming the positive staining intensity at a magnification of from about 20× to about 40×;
the Tumor Intensity Proportion Score (TIPS) comprises the number of CEACAM5 viable tumor or cancer cells staining at a 2+ or greater positive staining intensity divided by the total number of staining and non-staining viable tumor or cancer cells, multiplied by 100; or, a TIPS of about 40% or greater indicates a diagnostic status of the tissue sample; or, the TIPS is about 50% or greater, about 60% or greater, about 80% or greater, or about 90% or greater;
a TIPS of about 5% or greater indicates a diagnostic status of the tissue sample; or, a TIPS of about 5% or greater comprises the number of CEACAM5 viable tumor or cancer cells staining at a 2+ or greater positive staining intensity; or a TIPS of about 10% or greater comprises the number of CEACAM5 viable tumor or cancer cells staining at a 2+ or greater positive staining intensity;
a section or portion of the tissue sample is prepared on a slide, a microscope slide, or equivalent, and the section or portion of the tissue sample is stained on the slide;
the antibody comprises a monoclonal mouse anti-CEACAM5 antibody or a monoclonal rabbit anti-CEACAM5 antibody; or, the monoclonal mouse anti-CEACAM5 antibody comprises monoclonal mouse anti-CEACAM5 clone 769, or an antibody having a substantially similar affinity for CEACAM5 as a clone 769 antibody;
the tissue sample comprises a formalin-fixed, paraffin-embedded (FFPE) specimen; or, the FFPE specimen comprises a cancer specimen stained on an automated IHC platform;
the section of the tissue sample is prepared by a protocol comprising fixation in about 10% neutral buffered formalin for a time period of about 6 hours to about 72 hours;
the tumor or cancer is a non-squamous non-small cell lung cancer (nsNSCLC), a non-small cell lung cancer (NSCLC), an adenocarcinoma, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a stomach cancer, a colorectal cancer, a lung cancer, a colon cancer, a breast cancer, an ovarian cancer, an endometrial cancer, a cervical cancer, a prostate cancer, or a urothelial cancer;
the tissue sample is derived from a needle biopsy sample, a fine-needle aspirate, a cytology specimen, or a bone decalcification; and/or
a positive staining is determined using a bright-field light microscope, a microscope objective, a computer monitor and imaging software, or a combination thereof; and optionally, the imaging software comprises whole slide imaging software.

In alternative embodiments, provided are methods for diagnosing a tumor or a cancer by determining if a tissue sample is positive for expression of carcinoembryonic antigen-related cell adhesion molecule 5 (CEACAM5), comprising: determining a CEACAM5 diagnostic status in a tissue sample by a method as provided herein, for example, as described above, wherein a Tumor Intensity Proportion Score of about 50% or greater of tumor cancer cells having CEACAM5 cellular membrane staining at an intensity of 2+ or greater is diagnostically positive. In alternative embodiments, the tumor or cancer is a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer.

In alternative embodiments, provided are methods for treating or ameliorating a tumor or a cancer in a patient, comprising determining and scoring the amount of CEACAM5 in a tissue sample from the patient using a method as provided herein, for example, as described above, wherein if the tissue sample is determined or scored to have a high or a diagnostically positive CEACAM5 score, the patient is treated with a cancer therapeutic to which the patient is likely to respond favorably.

In alternative embodiments of methods as provided herein:
the tumor or cancer is a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer;
the cancer therapeutic comprises administration to the patient an anti-cancer drug or an anti-cancer therapy; and/or
the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (CAR) T-cell therapy.

In alternative embodiments, provided are kits comprising an antibody which specifically binds to CEACAM5 and CEACAM5 scoring guidelines as provided herein, for example, comprising a method as provided herein, for example, as described above.

In alternative embodiments, provided are methods for assessing the extent of CEACAM5 expression comprising:
contacting a sample or a portion thereof comprising cancer or tumor cells from an individual with an antibody or a portion thereof which specifically binds to CEACAM5; and
determining a Tumor Intensity Proportion Score (TIPS) by dividing the number of CEACAM5 staining viable tumor or cancer cells in the sample or portion thereof specifically bound by the antibody with the total number of staining and non-staining viable cancer or tumor cells and multiplying the result by 100, thereby obtaining the Tumor Intensity Proportion Score.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

Like reference symbols in the various drawings indicate like elements.

### DETAILED DESCRIPTION

In alternative embodiments, provided are immunohistochemistry (IHC) methods for determining and scoring the extent of expression of the protein carcinoembryonic antigen-related cell adhesion molecule 5, or CEACAM5 (also known as CD66e (Cluster of Differentiation 66e)), in a tissue sample. In alternative embodiments, provided are scoring methods to assess CEACAM5 expression in tumors or cancers such as non-squamous non-small cell lung cancer (nsNSCLC).

Alternative embodiments of IHC methods herein provide robust, reliable, standardized, reproducible and harmonized methods for assessing the extent of cellular membrane expression of CEACAM5, thus creating greater between-laboratory and between-study comparability, and allowing earlier valid applications of CEACAM5 in clinical practice. Embodiments of IHC methods herein provide for high quality staining and reliable diagnostic assessment.

Current immunohistochemistry scoring methods for CEACAM5 measure the percentages of cells in a tissue sample that exhibit whole membrane staining and luminal (polarized) membrane staining. These percentages of whole versus luminal staining are placed in separate scoring categories. The percentages are further divided into staining intensity bins. In order to determine a final CEACAM5 score relative to the cutoff, only staining at 2+ and 3+ intensities are considered. Separation of membrane expression level scoring into multiple categories and staining intensity bins adds complexity to the scoring process. The greater complexity of such scoring methods in turn adds to the time required to reach a diagnostic assessment. Pathologists can also vary in their individual determinations about the presence of or relative staining intensities of membrane and luminal staining patterns among the various cells in tissue samples, adding difficulty to reproducibility of scoring methods. Moreover, it can be confusing as to how to apply the scoring guidelines, since tumor morphology in some cancers is heterogeneous, including in non-squamous non-small cell lung cancer (nsNSCLC). There remains a need in the art for simpler and more efficient, as well as robust, reproducible, and accurate scoring methods to assess CEACAM5 expression in tissue samples.

Embodiments herein are directed to immunohistochemistry methods for determining and scoring the extent of cellular membrane expression of carcinoembryonic antigen-related cell adhesion molecule 5 (CEACAM5) in a tissue sample. In various embodiments, the method comprises: staining a tissue sample with an antibody which specifically binds to CEACAM5; determining a total number of viable tumor or cancer cells having CEACAM5 cellular membrane staining, and determining a total number of staining and non-staining viable tumor or cancer cells in at least a portion of a tissue sample, wherein a tumor or cancer cell is counted as positively stained with anti-CEACAM5 antibody if there is CEACAM5 cellular membrane staining at any intensity above a defined threshold; and determining a Tumor Intensity Proportion Score (TIPS), wherein the TIPS is the number of CEACAM5 staining viable tumor or cancer cells found in the tissue sample divided by the total number of staining and non-staining viable tumor or cancer cells, multiplied by 100. In various embodiments herein, the Tumor Intensity Proportion Score (TIPS) can also be referred to as a CEACAM5 positivity score. Such embodiments can provide not only robust, reliable, reproducible and accurate CEACAM5 scoring methods, but also scoring methods having greater simplicity and efficiency. Such embodiments can provide a considerable advantage for reliable and efficient diagnostic assessments.

In certain embodiments, the CEACAM5 cellular membrane staining comprises whole membrane staining, discontinuous membrane staining, partial membrane staining, complete membrane staining, punctate membrane staining, linear membrane staining, luminal membrane staining, apical membrane staining, basal membrane staining, lateral membrane staining, basolateral membrane staining, high cytoplasmic staining at a 2+ or greater positive staining intensity, high cytoplasmic staining at a 3+ positive staining intensity, or a combination thereof. In certain embodiments, the total number of viable tumor or cancer cells comprises luminal cells, glandular luminal cells, intraluminal cells, multiple layers of cells, cells having signet ring morphology, high cytoplasmic staining cells, cells with intracytoplasmic lumina, or a combination thereof. In certain embodiments, the total number of viable tumor or cancer cells counted as positively stained comprises all glandular luminal cells having a cellular membrane associated with the positively staining lumen. Such embodiments can provide an advantage of including all observed patterns and levels of cellular membrane staining, cytoplasmic staining, and all types of viable tumor or cancer cells in the determination of a Tumor Intensity Proportion Score. Such embodiments can provide a benefit of simplified IHC scoring methods for scoring cellular membrane expression of CEACAM5 in tissue samples.

In certain embodiments, the tissue sample comprises at least about 100 cells. In certain embodiments, the tissue sample comprises at least about 200 cells. In certain embodiments, the tissue sample comprises at least about 500 cells.

In some embodiments, a tumor or cancer cell counted as positively stained excludes tumor or cancer cells having cytoplasmic staining, staining of normal or non-neoplastic structures, staining nonviable tumor or cancer cells, necrotic cells, cellular debris, stromal staining, or edge artifact staining on a periphery of the tissue sample. In certain embodiments, cytoplasmic staining comprises cytoplasmic staining at a 2+ or lesser positive staining intensity. Such embodiments can provide advantages of increasing the accuracy and reproducibility of CEACAM5 scoring methods by excluding stained cells having certain structures or characteristics from the Tumor Intensity Proportion Score (TIPS) determination.

In certain embodiments, the defined threshold comprises a 2+ or greater positive staining intensity. In certain embodiments, the defined threshold comprises a 2+ positive or a 3+ positive staining intensity evaluated at a magnification of from about 4× to about 10×. In certain embodiments, the method further comprises confirming the positive staining intensity at a magnification of from about 20× to about 40×. In certain embodiments, the Tumor Intensity Proportion Score comprises the number of CEACAM5 viable tumor or cancer cells staining at a 2+ or greater positive staining intensity divided by the total number of staining and non-staining viable tumor or cancer cells, multiplied by 100. Such embodiments can provide a benefit of versatility in the staining intensity of the defined threshold.

In certain embodiments, a Tumor Intensity Proportion Score (TIPS) of about 40% or greater indicates a diagnostic status of the tissue sample. In certain embodiments, the TIPS is about 50% or greater, about 60% or greater, about 80% or greater, or about 90% or greater. In certain embodiments, a TIPS of about 5% or greater indicates a diagnostic status of the tissue sample. In certain embodiments, a TIPS of about 1% or greater indicates a diagnostic status of the tissue sample. In certain embodiments, a TIPS of about 5% or greater comprises the number of CEACAM5 viable tumor or cancer cells staining at a 2+ or greater positive staining intensity; or a TIPS of about 10% or greater comprises the number of CEACAM5 viable tumor or cancer cells staining at a 2+ or greater positive staining intensity. Such embodiments can provide a benefit of accuracy in a diagnostic status determination related to CEACAM5 expression.

In alternative embodiments, provided are IHC methods comprising a simplified scoring system for membrane staining at ≥2+ intensity threshold, and provided is guidance for dealing with different morphological patterns of luminal membrane staining. Inclusion of intraluminal tumor cells in the CEACAM5 assessment when viability and tumor cell identity can be confirmed. Guidance for evaluating tumor cells with strong (3+) cytoplasmic staining that may obscure membrane staining, when tumor cells with unambiguous membrane staining are present within the same 10× field of view.

In alternative embodiments, methods and kits as provided herein are used for in vitro diagnostic uses. In alternative embodiments, the CEACAM5 IHC as provided herein is an immunohistochemical (IHC) assay using an anti-CEACAM5 antibody such as the monoclonal mouse anti-CEACAM5 clone 769, in the detection of a CEACAM5 protein in formalin-fixed, paraffin-embedded (FFPE) tissue samples such as lung carcinoma tissue sample. In alternative embodiments, the EnVision FLEX™ visualization system on Dako OMNIS™ is used.

In alternative embodiments, the CEACAM5 IHC as provided herein is used as an aid in identifying patients with a tumor or a cancer, for example, a non-squamous non-small cell lung cancer (nsNSCLC), a non-small cell lung cancer (NSCLC), an adenocarcinoma, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a stomach cancer, a colorectal cancer, a lung cancer, a colon cancer, a breast cancer, an ovarian cancer, an endometrial cancer, a cervical cancer, a prostate cancer, or a urothelial cancer.

In alternative embodiments, methods for assessing the extent of CEACAM5 expression comprise: contacting a sample or a portion thereof comprising cancer or tumor cells from an individual with an antibody or a portion thereof which specifically binds to CEACAM5; and determining a Tumor Intensity Proportion Score by dividing the number of CEACAM5 staining viable tumor or cancer cells in the sample or portion thereof specifically bound by the antibody with the total number of staining and non-staining viable cancer or tumor cells and multiplying the result by 100, thereby obtaining the Tumor Intensity Proportion Score.

In certain embodiments, a section or portion of the tissue sample is prepared on a slide, a microscope slide, or equivalent, and the section or portion of the tissue sample is stained on the slide. In certain embodiments, the tissue sample comprises a formalin-fixed, paraffin-embedded (FFPE) specimen. In certain embodiments, the FFPE specimen comprises a cancer specimen stained on an automated IHC platform. In certain embodiments, the section of the tissue sample is prepared by a protocol comprising fixation in about 10% neutral buffered formalin for a time period of about 6 hours to about 72 hours. In certain embodiments, the tissue sample is derived from a needle biopsy sample, a fine-needle aspirate, a cytology specimen, or a bone decalcification.

In certain embodiments, the antibody comprises a monoclonal mouse anti-CEACAM5 antibody or a monoclonal rabbit anti-CEACAM5 antibody. In some embodiments, the monoclonal mouse anti-CEACAM5 antibody comprises monoclonal mouse anti-CEACAM5 clone 769, or an antibody having a substantially similar affinity for CEACAM5 as a clone 769 antibody.

In certain embodiments, the tumor or cancer is a non-squamous non-small cell lung cancer (nsNSCLC), a non-small cell lung cancer (NSCLC), an adenocarcinoma, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a stomach cancer, a colorectal cancer, a lung cancer, a colon cancer, a breast cancer, an ovarian cancer, an endometrial cancer, a cervical cancer, a prostate cancer, or a urothelial cancer.

In certain embodiments, the positive staining is determined using a bright-field light microscope, a microscope objective, a computer monitor and imaging software, or a combination thereof. In some aspects, the imaging software comprises whole slide imaging software.

### Embodiments of Methods for Diagnosing a Tumor or a Cancer

Embodiments herein provide methods for diagnosing a tumor or a cancer by determining if a tissue sample is positive for expression of carcinoembryonic antigen-related cell adhesion molecule 5 (CEACAM5). In various aspects, the method comprises: determining a CEACAM5 diagnostic status in a tissue sample by an IHC method for determining the extent of cellular membrane expression of CEACAM5 as provided herein, wherein a Tumor Intensity Proportion Score of about 50% or greater of tumor cancer cells having CEACAM5 cellular membrane expression at an intensity of 2+ or greater is diagnostically positive. In certain embodiments, the tumor or cancer is a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer.

### Embodiments of Methods for Treating Cancers and Tumors

Provided are methods for treating or ameliorating a tumor or a cancer in a patient, comprising determining and scoring the amount of CEACAM5 in a tissue sample from the patient using a method as provided herein, wherein if the tissue sample is determined or scored to have a high or a diagnostically positive CEACAM5 score, the patient is treated with a cancer therapeutic or an anti-cancer therapy to which the patient is likely to respond favorably. The tumor or cancer can be a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer.

In certain embodiments, the cancer therapeutic comprises administration to the patient an anti-cancer drug or an anti-cancer therapy. In alternative embodiments, the anti-cancer treatment or therapy comprises: surgery such as cyberknife therapy; chemo-embolization; an ablation technique such as radiofrequency ablation (RFA), cryoablation and/or microwave ablation; and/or, radiation therapy such as stereotactic body radiation therapy.

In certain embodiments, the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (CAR) T-cell therapy. In alternative embodiments, the anti-cancer treatment or therapy comprises: a tyrosine kinase inhibitor (optionally erlotinib (or TARCEVA™), gefitinib (or IRESSA™), afatinib (or GILOTRIF™), or osimertinib (TAGRISSO™)); necitumumab (or PORTRAZZA™), pembrolizumab (or KEYTRUDA™), nivolumab (or OPDIVO™), ipilimumab (YERVOY™), cetuximab (or ERBITUX™), cisplatin (or PLATINOL™) or carboplatin (or PARAPLATIN™).

In alternative embodiments, the anti-cancer treatment or therapy comprises use of an anti-cancer drug that can comprise an antibody that specifically or substantially binds to the cancer or tumor, wherein the antibody is conjugated to a cytotoxic agent, and optionally the cytotoxic agent comprises a radionuclide (optionally Yttrium-90, Iodine-131, Lutetium-177, Radium-223 chloride, strontium-89 chloride or samarium-153 EDTMP), diphtheria toxin, pseudomonas exotoxin A, denileukin diftitox, moxetumomab pasudotox, calicheamicin or N-acetyl-γ-calicheamicin, emtansine or DM1, maytansine or derivatives thereof, SN-38 (or 7-Ethyl-10-hydroxycamptothecin), or auristatin or monomethyl auristatin E (MMAE).

### Immunohistochemistry

In alternative embodiments, immunohistochemistry methodologies and/or reagents used with methods and products of manufacture or kits as provided herein can include or comprise or comprise use of any IHC protocol, IHC armamentarium, device and/or image or data analysis system, for practicing IHC or IHC reagents known in the art, for example, as described in U.S. Pat. No. 10,634,590 (describing a slide holder assembly fixture for use in IHC); U.S. Pat. No. 10,565,479 (describing methods for identifying blurred areas in digital images of stained tissue); U.S. Pat. No. 10,564,076 (describing systems for analytical (or IHC) sample preparation); U.S. Pat. No. 10,551,395 (describing an automated histological staining system); U.S. Pat. No. 10,551,378 (describing a tissue staining method); U.S. Pat. No. 10,504,224 (describing a digital tissue image analysis system for IHC); U.S. Pat. No. 10,501,777 (describing simultaneous, multiplexed detection and quantification of protein expression in IHC); U.S. Pat. No. 10,488,340 (describing method for extracting an image of a target fluorophore in a biological material); U.S. Pat. No. 10,453,195 (describing methods of detecting tissue areas of interest using digital pathology imaging); U.S. Pat. No. 10,438,381 (describing devices, systems and methods for generating a digital image of a tissue section); U.S. Pat. No. 10,430,943 (describing methods and programs for automated nuclei area/number estimation for IHC image analysis); U.S. Pat. No. 10,416,176 (describing methods for processing specimens in an automated histological staining system); U.S. Pat. No. 10,393,633 (describing methods for processing and inhibiting the degradation of an IHC sample); U.S. Pat. No. 10,217,011 (describing handling of IHC slides); U.S. Pat. No. 10,209,165 (describing automated or semi-automated methods for assessing the quality of staining of a specimen containing cells); U.S. Pat. No. 10,126,216 (describing methods for fixing tissue samples for IHC); U.S. Pat. Nos. 9,423,322; 8,515,683 (describing methods and systems for automated detection of immunohistochemical (IHC) patterns); U.S. Pat. No. 10,816,443 (describing automated batch stainers for staining biological specimens on microscope slides); or, U.S. patent application publication nos: US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); US 2019/0293637 A1 (methods and systems for quantitative immunohistochemistry (IHC) of a target protein molecule); US 2019/0080450 A1 (describing an automated determination of the staining quality of an IHC stained biological sample); or, US 2020/0316589 A1 (describing a multi-well solid support vessel for the processing and testing of fixed biological materials).

In alternative embodiments, the antibodies, antigen binding fragments thereof, or monomeric or dimeric antigen binding proteins as provided herein (including for example synthetic or recombinant forms) used in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase), for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-1-O-carboxylic acid; 7-(diethylamino)coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester; DY-405; Alexa Fluor 405 succinimidyl ester; Cascade Yellow succinimidyl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide; Alexa Fluor 430 succinimidyl ester; Dabcyl succinimidyl ester; NBD chloride/fluoride; QSY 35 succinimidyl ester; DY-485XL; Cy2 succinimidyl ester; DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505; Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL; DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester; DY-555; DYQ-1; DY-556; Cy3 succinimidyl ester; DY-547; DY-549; DY-550; Alexa Fluor 555 succinimidyl ester; Alexa Fluor 546 succinimidyl ester; DY-548; BODIPY 558/568 C3 succinimidyl ester; Rhodamine red-X succinimidyl ester; QSY 7 succinimidyl ester; BODIPY 564/570 C3 succinimidyl ester; BODIPY 576/589 C3 succinimidyl ester; carboxy-X-rhodamine (ROX); succinimidyl ester; Alexa Fluor 568 succinimidyl ester; DY-590; BODIPY 581/591 C3 succinimidyl ester; DY-591; BODIPY TR-X succinimidyl ester; Alexa Fluor 594 succinimidyl ester; DY-594; carboxynaphthofluorescein succinimidyl ester; DY-605; DY-610; Alexa Fluor 610 succinimidyl ester; DY-615; BODIPY 630/650-X succinimidyl ester; erioglaucine; Alexa Fluor 633 succinimidyl ester; Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701; DY-704; DY-700; DY-730; DY-731; DY-732; DY-734; DY-750; Cy7 succinimidyl ester; DY-749; DYQ-4; Cy7.5 succinimidyl ester; 7-diethylaminocoumarin-3-carboxylic acid; succinimidyl ester; Dabsyl sulfonyl chloride; fluorescein isothiocyanate (FITC) carboxy succinimidyl ester (DY-495); Rhodamine Green carboxylic acid succinimidyl ester (DY-505); eosin isothiocyanate (EITC); 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye as described in U.S. patent application no. US 2019/0018018 A1. 3,3′-Diaminobenzidine (DAB) also can be used.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, CEACAM5), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 μm to 5 μm. The sections or slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make a CEACAM5 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using EnVision FLEX Mini Kit, High pH, which is a high-sensitivity visualization system intended for use in IHC together with Dako AUTOSTAINER™ instruments; this dual link system detects primary mouse and rabbit antibodies and the reaction is visualized by 3,3-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein, including for example, monoclonal mouse anti-CEACAM5 antibodies such as clone 769 anti-CEACAM5 antibodies, and/or reagents for practicing IHC, including for example reagents as described herein, see Example 1, below; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK).

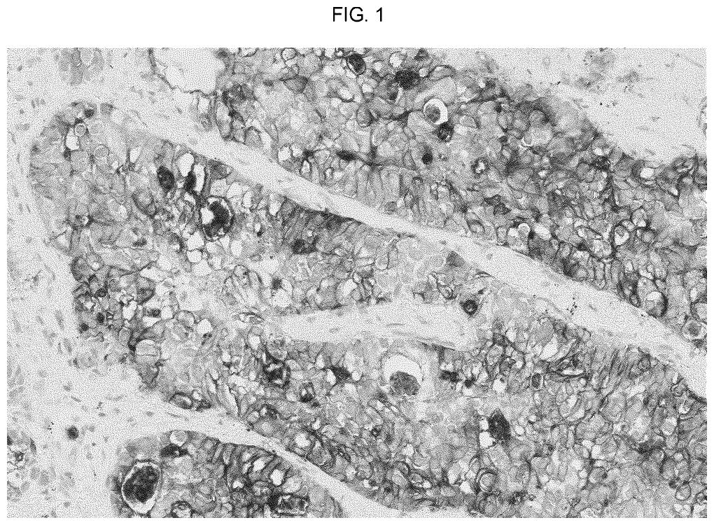

FIG. 1 illustrates a representative image of CEACAM5 staining in non-squamous NSCLC using a CEACAM5 IHC (antibody clone) 769 assay, as described in further detail in Example 1, below.

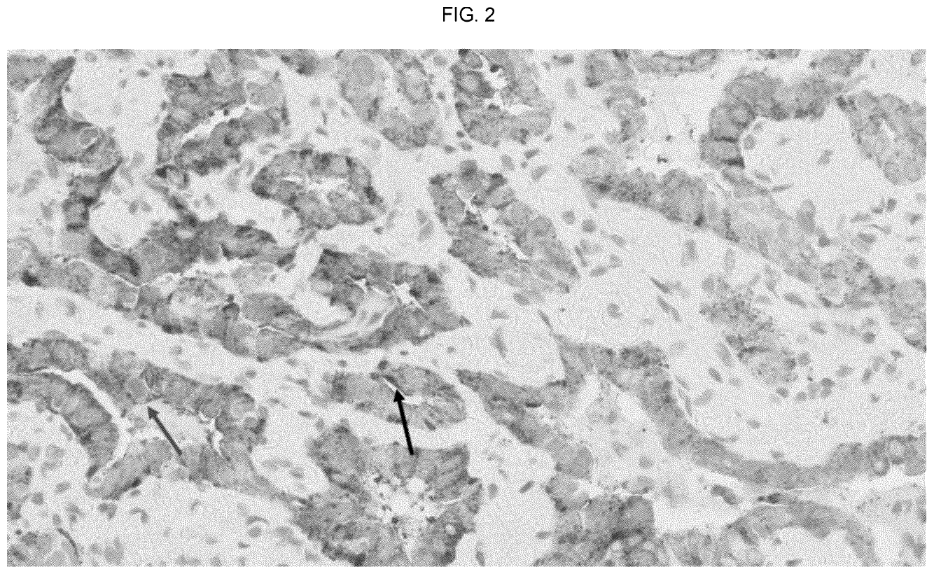

FIG. 2 illustrates a representative image of positive staining examples: Punctate (red arrow, or arrow at left edge image) and linear (black arrow, or arrow at middle of image) membrane staining at greater than ≥2+ intensity are considered positive; the 40× image is shown here for detailed illustration, as described in further detail in Example 1, below.

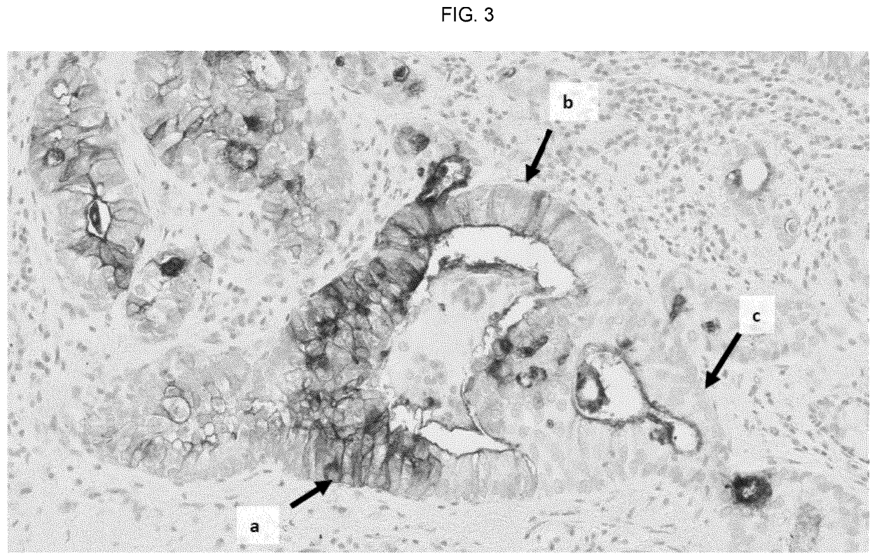

FIG. 3 of partial and complete membrane staining: arrow “a” indicates complete membrane staining; arrow “b” indicates partial, basolateral and apical (luminal) membrane staining; and arrow “c” indicates partial, apical (luminal) membrane staining (alveolar pattern), as described in further detail in Example 1, below.

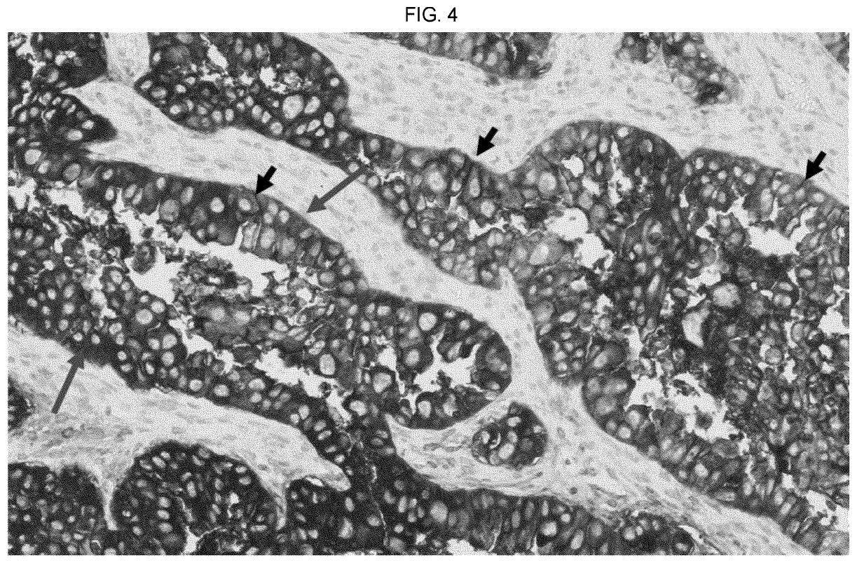

FIG. 4 illustrates CEACAM5 scoring: red (or longer) arrows indicate areas where high cytoplasmic staining without distinguishable membrane staining is observed, and the cells are scored as positive for CEACAM5 because tumor cells within a 10× objective field (black (or shorter) arrows) have discernible membrane staining and indicate that the area is highly positive for CEACAM5, as described in further detail in Example 1, below.

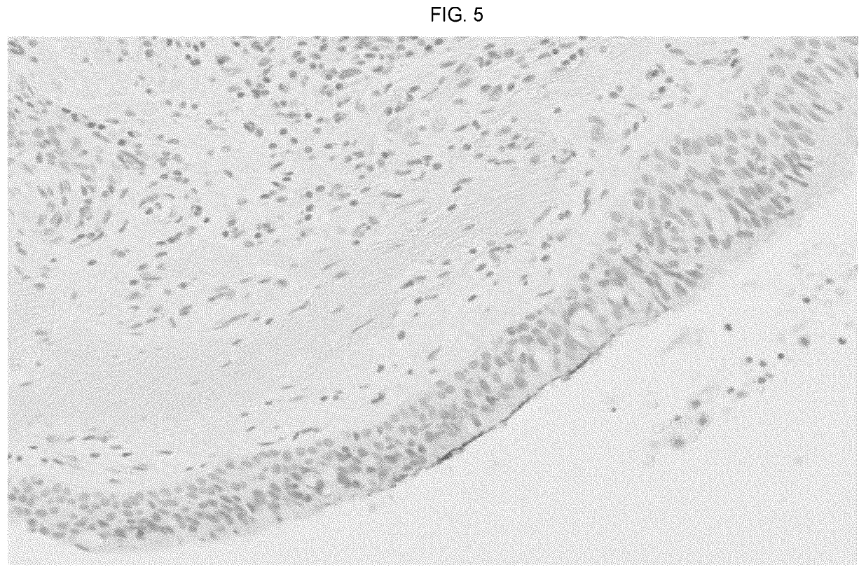

FIG. 5 illustrates an exemplary benign tissue staining example: staining of normal (non-neoplastic) tissue, as shown here, as described in further detail in Example 1, below.

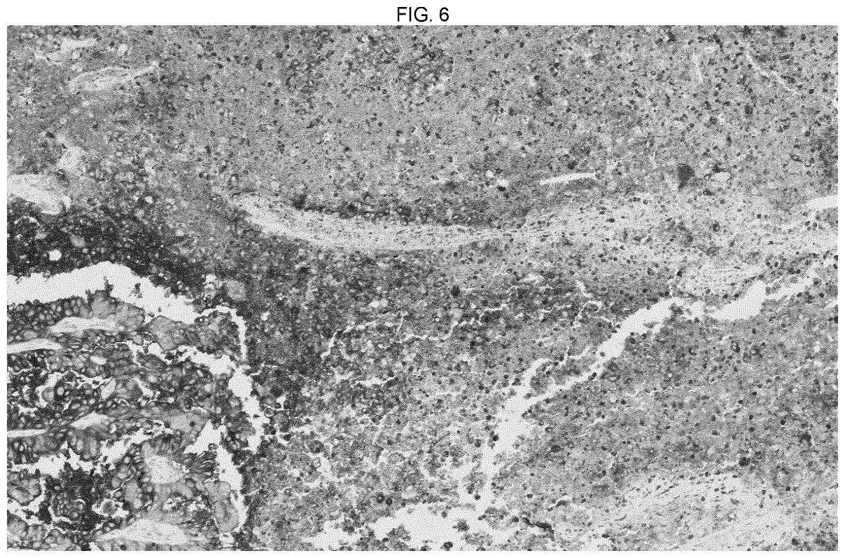

FIG. 6 illustrates an image demonstrating that staining may occur in necrotic tissue and at a range of intensities, as described in further detail in Example 1, below.

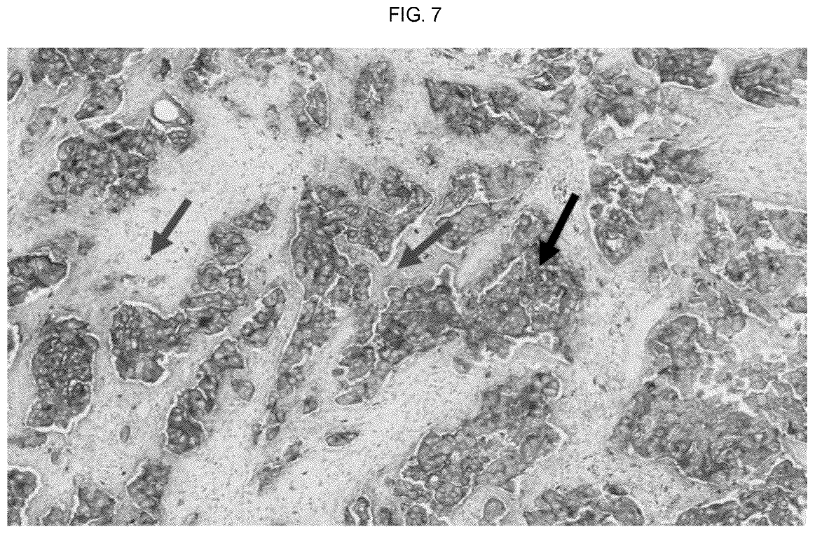

FIG. 7 illustrates an image demonstrating that only tumor cells (black (or longer) arrow) are scored for CEACAM5 positivity, and any surrounding stroma (red (shorter) arrows) showing CEACAM5 staining is excluded from the percent score of CEACAM5 staining protocols as provided herein, and not considered as non-specific background staining, as described in further detail in Example 1, below.

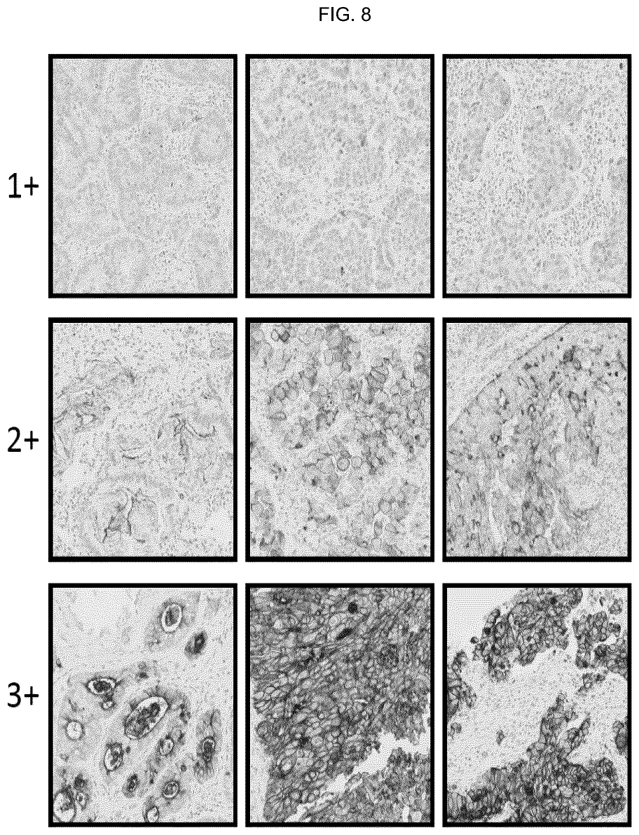

FIG. 8 illustrates images of 1+ (upper row), 2+ (middle row), 3+ (lower row) staining intensities using CEACAM5 scoring protocols as provided herein, as described in further detail in Example 1, below.

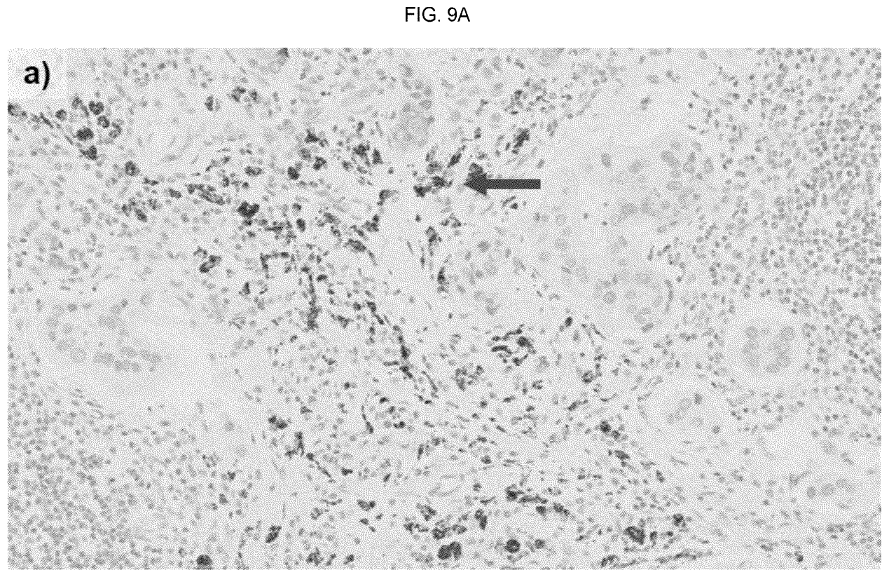

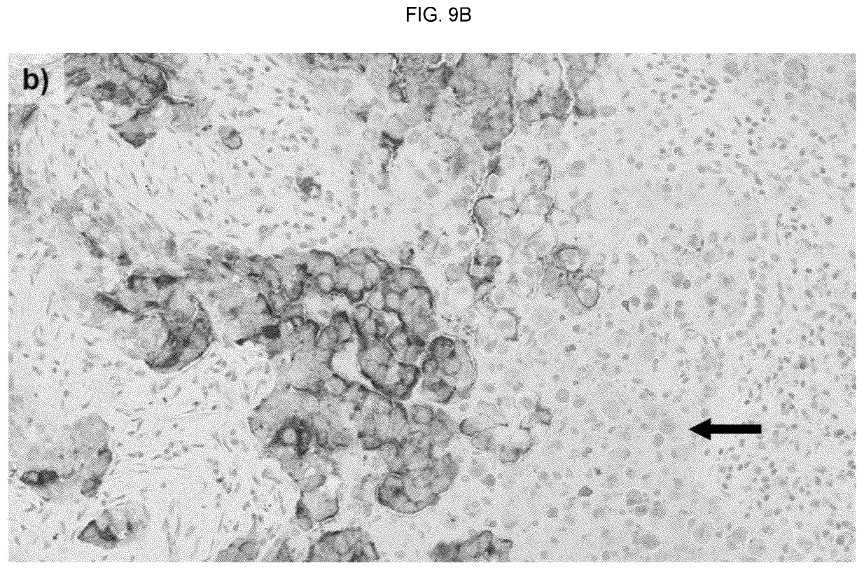

FIG. 9A-B illustrate images demonstrating that pigments that may be present in specimens, for example, pigments such as anthracotic pigment (FIG. 9A arrow) and hemosiderin (FIG. 9B arrow) are distinguishable from DAB precipitate at moderate to high magnification and should not be included in scoring for CEACAM5 positivity using CEACAM5 scoring protocols as provided herein, as described in further detail in Example 1, below.

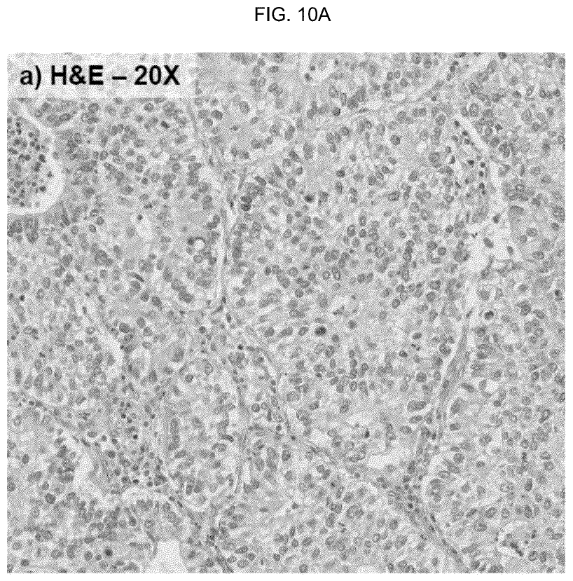

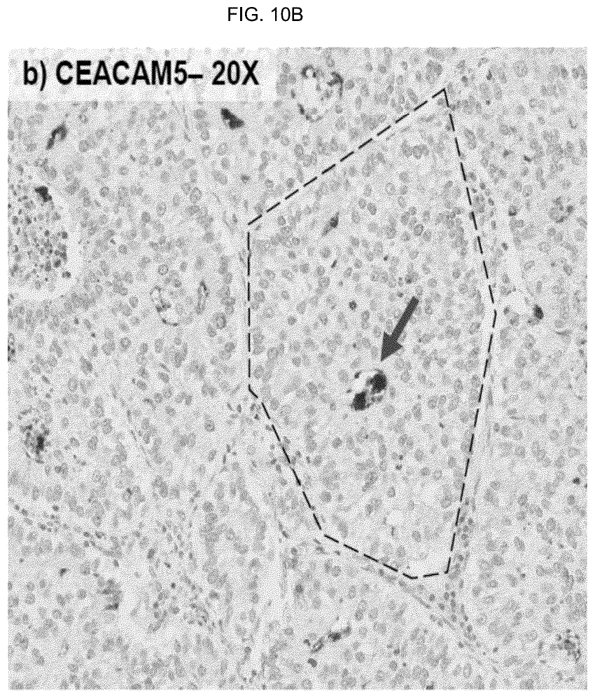

FIG. 10A-B illustrate images of luminal staining details and nested patterns: FIG. 10A show H&E (hematoxylin and eosin) staining at 20×; and FIG. 10B shows CEACAM5 positive apical membrane staining at 20× in the luminal portion of the nest, as described in further detail in Example 1, below.

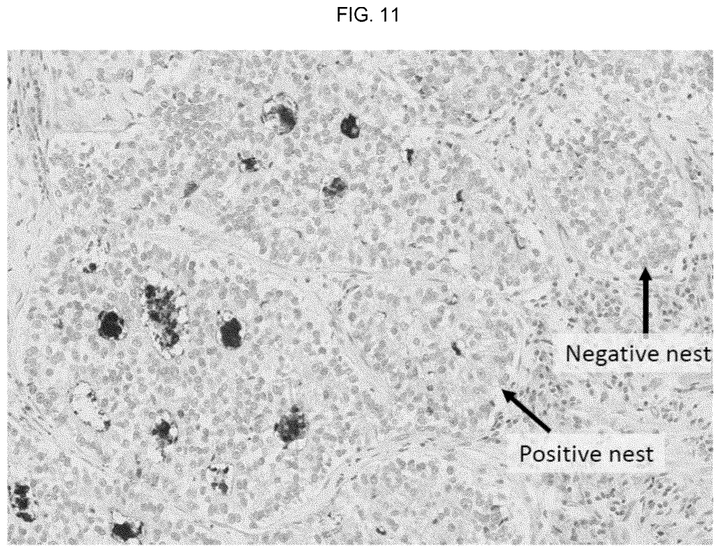

FIG. 11 illustrates an image of luminal staining details and nested patterns; arrows indicate nests that are positive or negative for CEACAM5, as described in further detail in Example 1, below.

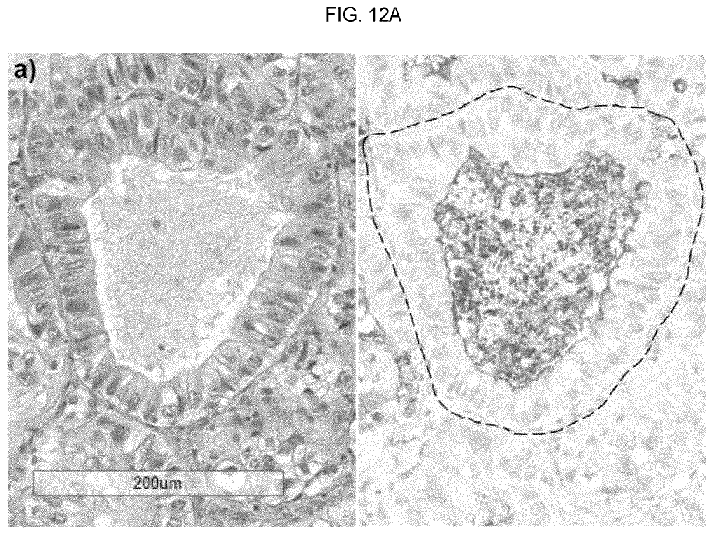

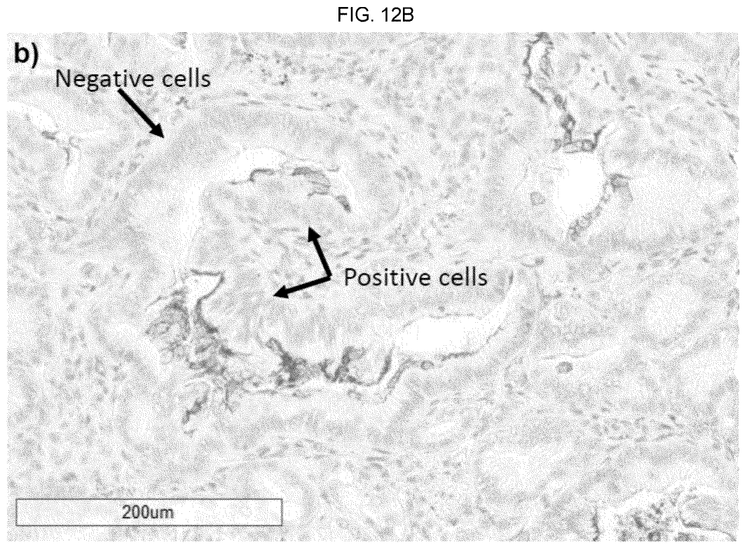

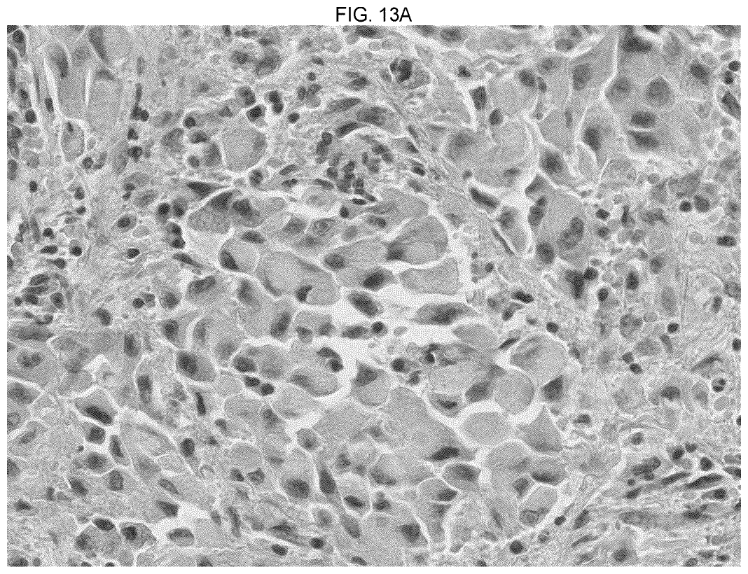

FIG. 12A-B illustrate images of luminal staining details and alveolar patterns: FIG. 12A: all cells in the outlined alveolar structure are positive; and FIG. 12B: the alveolar structure at the center of this panel contains positive and negative cells, as described in further detail in Example 1, below.

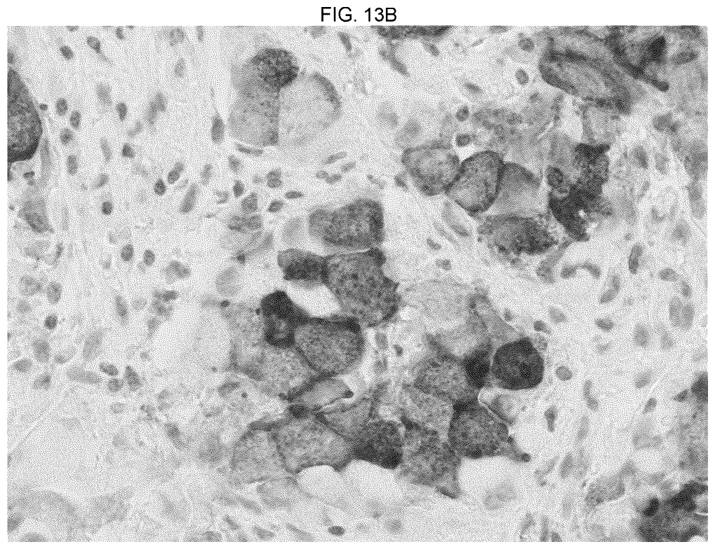

FIG. 13A-B illustrate images of H&E staining (FIG. 13A) and CEACAM5 staining (FIG. 13B) of cells with signet ring morphology, as described in further detail in Example 1, below.

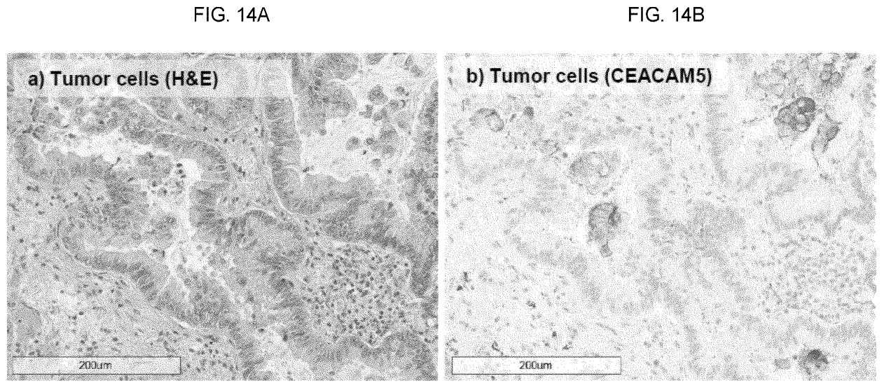

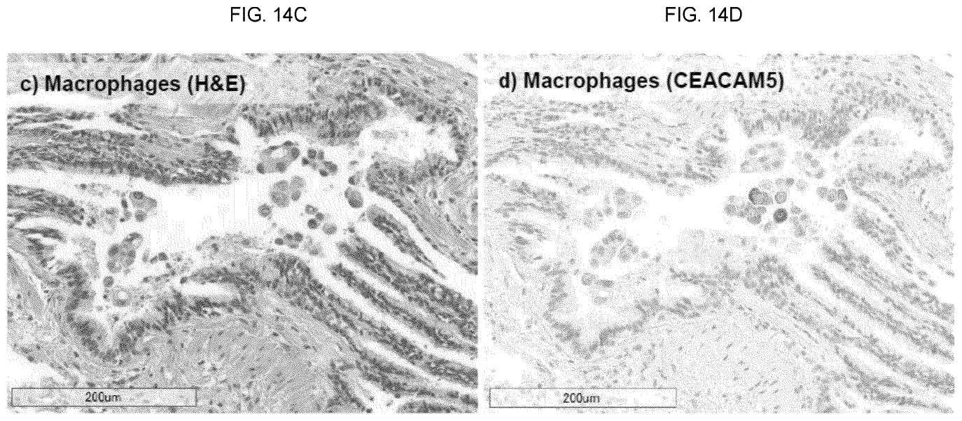

FIG. 14A-D illustrate images of viable tumor cells in intraluminal spaces (FIG. 14A; FIG. 14B) should be scored using CEACAM5 scoring protocols as provided herein if viability can be confirmed: FIG. 14A is H&E staining of tumor cells, and FIG. 14B is CEACAM5 staining of tumor cells; and FIG. 14C-D: Macrophages and non-viable tumor cells should not be scored, FIG. 14C is H&E staining, and FIG. 14D is CEACAM5 staining, as described in further detail in Example 1, below.

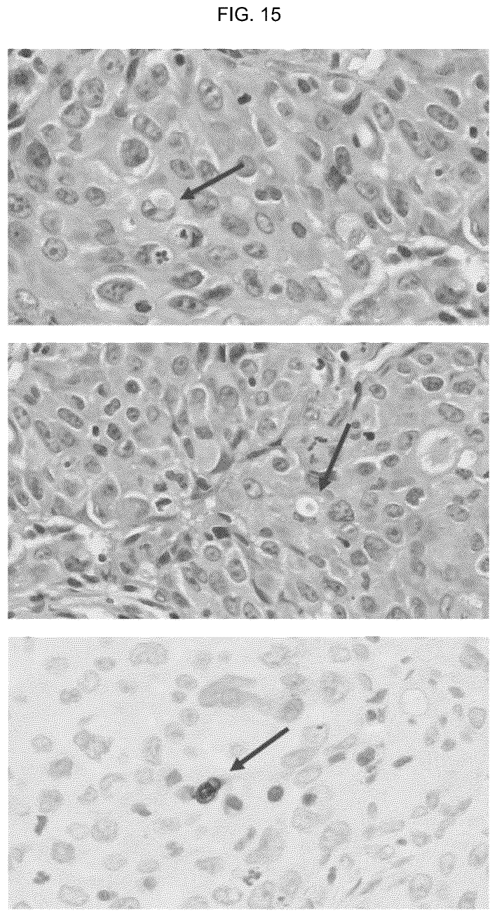

FIG. 15 illustrates images of intracytoplasmic lumina (arrows) staining demonstrating that CEACAM5 may be present, as described in further detail in Example 1, below.

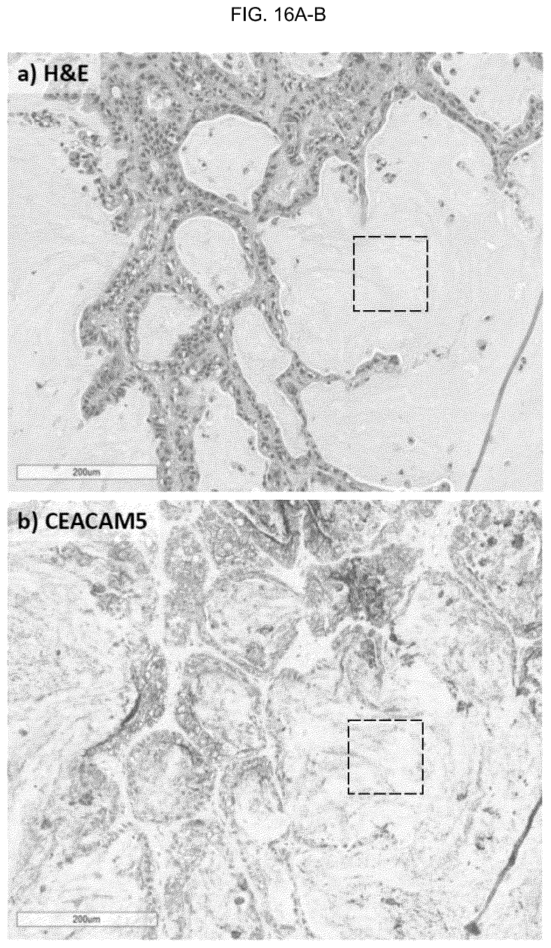

FIG. 16A-B illustrate images demonstrating that mucin can stain at high intensity (H&E staining FIG. 16A, and CEACAM5 staining FIG. 16B); example mucin staining shown in boxed area, as described in further detail in Example 1, below.

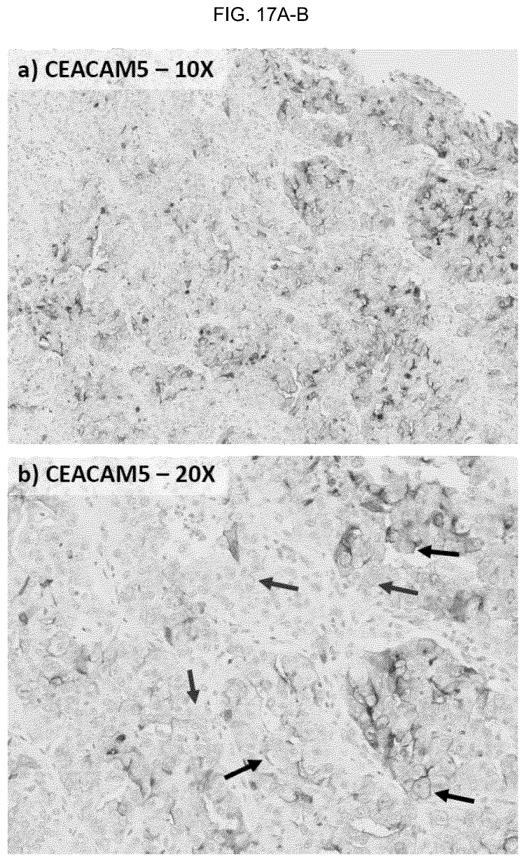

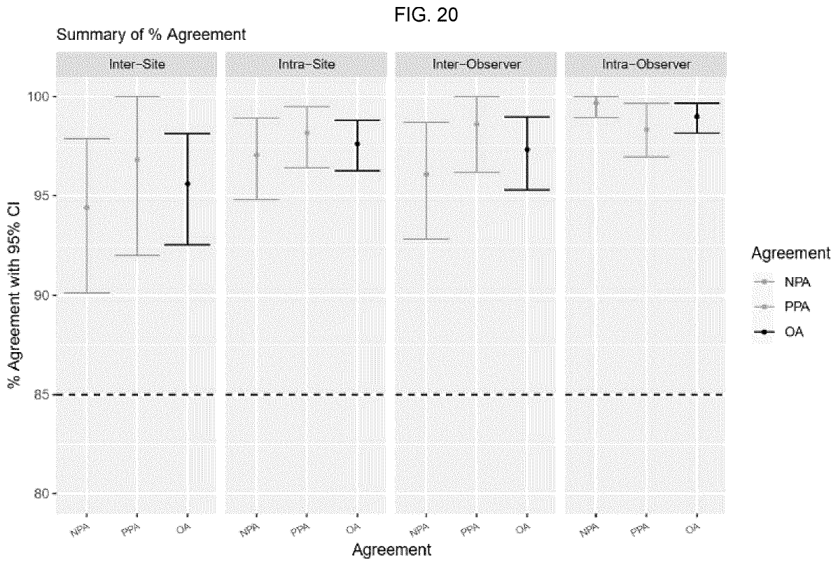

FIG. 17A-B illustrate positive control tissue images: non-squamous NSCLC tissue at 10× (FIG. 17A) and 20× (FIG. 17B) demonstrating a range of weak (1+, red arrows) to moderate (2+, black arrows) intensity membrane staining using CEACAM5 scoring protocols, as described in further detail in Example 1, below.

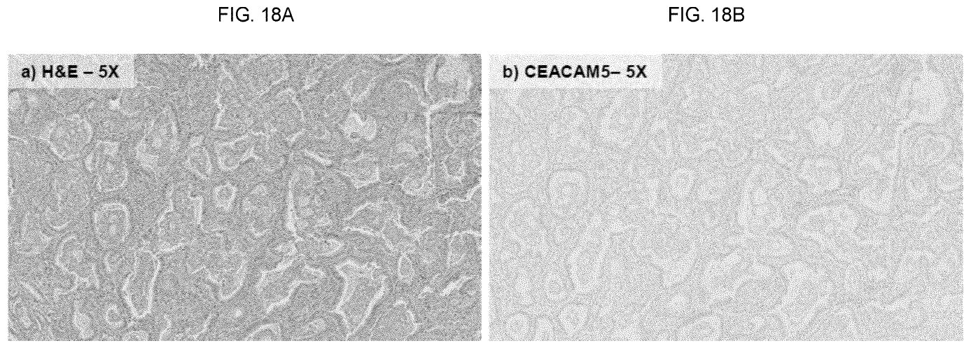

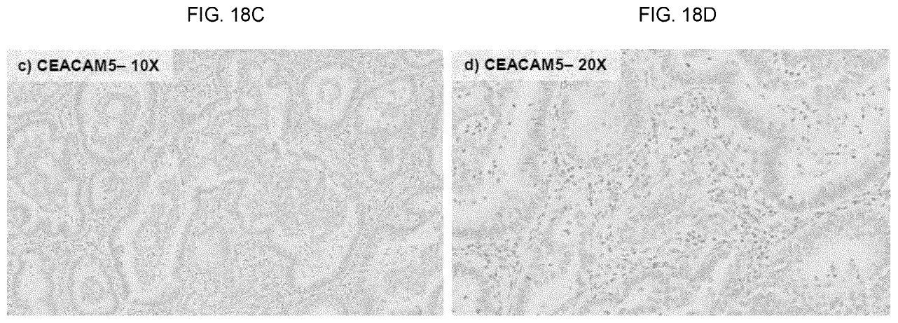

FIG. 18A-D illustrate negative control tissue images: FIG. 18A: H&E stain of non-squamous NSCLC; FIG. 18B: a low magnification objective can be used to initially scan tissue and observe any CEACAM5 staining; FIG. 18C: a higher magnification of 10× can be used to confirm if there is any staining; and, FIG. 18D: a 20× objective can be used to visualize if there is any specific membrane staining at the 1+ intensity, as described in further detail in Example 1, below

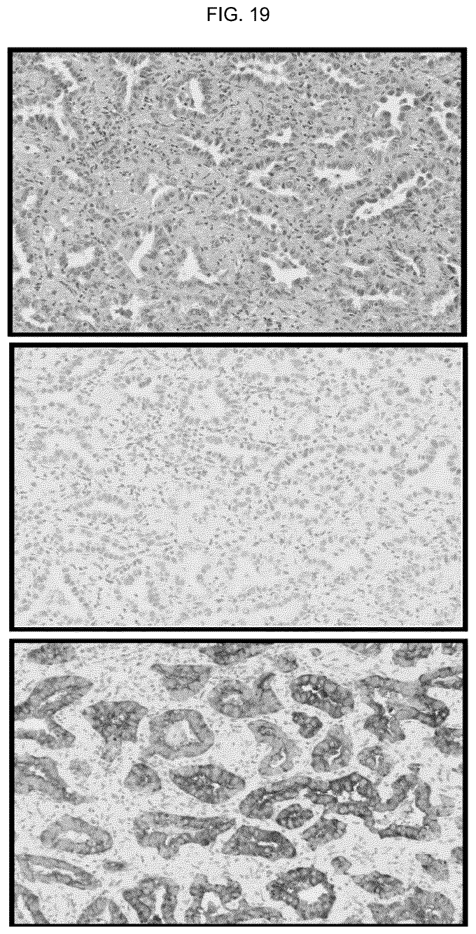

FIG. 19, top panel, illustrates an image of an H&E (hematoxylin and eosin) stain of a tissue specimen, which is evaluated first by H&E to assess tissue histology and preservation quality;

FIG. 19, middle panel, illustrates a negative control reagent slide, this is used to evaluate non-specific staining and allow interpretation of a CEACAM5 stained slide; and

FIG. 19, lower panel, illustrates CEACAM5 staining, which is assessed and, using CEACAM5 scoring protocols as provided herein, is considered positive staining if it is complete and/or partial plasma membrane staining at greater than or equal to (≥) 2+ intensity, as described in further detail in Example 1, below.

FIG. 20 illustrates a table summarizing the results of a study using CEACAM5 staining protocols as provided herein, as described in Example 2, below.

### Example 1: CEACAM5 IHC

This example demonstrates use of CEACAM5 staining protocols as provided herein.

Provided here are exemplary scoring guidelines for evaluation of CEACAM5 expression in non-squamous non-small cell lung cancer (non-squamous NSCLC) specimens that have been stained with the mouse monoclonal CEACAM5 antibody, designated mouse mAb clone 769. This antibody is provided in CEACAM5 IHC 769 assay for the identification of CEACAM5 expression in formalin-fixed, paraffin-embedded (FFPE) non-squamous NSCLC specimens using the Dako Omnis automated staining system.

Assessment of CEACAM5 IHC Staining Patterns in Non-Squamous Non-Small Cell Lung Cancer (Non-Squamous NSCLC) FFPE Tissues. The protocol-specific Pathology Report Form (PRF) is completed for each specimen evaluated (including “Not Evaluable” and “Indeterminate” as described below).

### CEACAM5 Scoring Summary

### Definition of Positive Staining

CEACAM5 positive staining is defined as any partial or complete tumor cell plasma membrane staining at ≥2+ intensity. Membrane staining may be linear or punctate. Staining may appear as complete or partial staining involving the basal, lateral, or basolateral aspects of the membrane, or as apical staining of the luminal aspect of the cell.

Representative staining examples are described below, see FIG. 1 to 19.

CEACAM5 percent positive score is determined by the following formula:

$$\% CEACAM 5 positive = 100 \times \frac{\begin{matrix}\# of tumor cells expressing CEACAM 5 \\ membrane staining at \geq 2 + intensity\end{matrix}}{Total \# of viable tumor cells present in the section}$$

A minimum of 100 evaluable and viable tumor cells must be present in the CEACAM5 stained slide to determine the percentage of stained cells.

Overall CEACAM5 diagnostic status is determined according to the following criteria:
The specimen is considered to be diagnostically positive if ≥X % of the tumor cells exhibit CEACAM5 positive staining at ≥2+ intensity, where X is the predetermined cut-off.
The specimen is considered to be diagnostically negative if <X % of the tumor cells exhibit CEACAM5 positive staining at ≥2+ intensity, where X is the predetermined cut-off.

For adenocarcinomas displaying glandular lumina, CEACAM5 partial plasma membrane staining may appear as apical membrane staining of the luminal aspect of the cell. In the case of luminal staining at ≥2+ intensity, all cells associated with the structure that may have membrane associated to the positive lumen will be considered positive.

### Definition of Negative Staining

Negative staining is defined as absence of membrane staining or any membrane staining of the tumor cells at <2+ intensity.

Included in scoring:
Partial or complete, punctate membrane staining and linear membrane staining at ≥2+

intensity is considered positive.
All viable tumor cells on a slide are evaluated for both intensity and frequency of positive staining.

Cell types and staining patterns to exclude from stain scoring:
Cytoplasmic staining is generally not considered as positive staining; however, positivity may be inferred for tumor cells with high cytoplasmic staining (3+) when positive membrane staining is identified in other tumor cells present within a 10× objective field of the high cytoplasmic staining cells. More detail and examples are provided in Section 2.1 and FIG. 4.
Staining of normal, non-neoplastic structures (FIG. 5)
Sloughed non-viable tumor cells
Necrotic cells and cellular debris (FIG. 6)
Stromal staining (FIG. 7)
Edge artifact staining on periphery of tissue specimen

### Scoring of Non-Squamous NSCLC

The staining intensity is scored using the following scale (Table 1):

TABLE 1
Staining Intensity Scale

| | | Staining intensity considered |
|---|---|---|
| Intensity Scale | Staining Intensity | positive for CEACAM5? |
| 0 | No staining | No |
| 1+ | Weak staining | No |
| 2+ | Moderate staining | Yes |
| 3+ | Strong staining | Yes |

Examples of CEACAM5 Staining at Each Intensity are Shown in FIG. 8.

CEACAM5 positive tumor staining at ≥2+ is scored using the 4×-10× microscope objectives. The 20× objective may be used to identify and/or confirm 1+ and punctate and/or delicate 2+ membrane staining intensity as well as 2+ intracytoplasmic lumina, which may not be apparent at the 4× and 10× objectives. The 20×-40× objectives may be used to confirm membrane staining localization if needed. The 40× objective should not be routinely used for scoring (i.e., to assess intensity and percent positivity).

This assay is being evaluated for non-squamous NSCLC tissue samples. H&E stained slides should accompany every CEACAM5 stained sample to allow proper confirmation of the presence of tumor and histological diagnosis, assess tumor viability, and to allow distinctions from non-tumor portions of the slide which should be excluded from the assessment. H&E and NCR stained slides may also assist with identifying intrinsic pigments (due to intrinsic anthracosis, hemosiderin pigment, etc.; see for example FIG. 9).

CEACAM5 expression patterns observed in non-squamous NSCLC:
Luminal Staining: Two morphological patterns of structures displaying luminal (apical membrane) staining may be observed in lung adenocarcinomas: nested and alveolar patterns. When luminal staining at 2+ intensity is observed in either pattern, all cells within the structure that may have membrane associated to the positive lumen should be considered positive.
Nested pattern: Solid nests delimited by stroma and containing multiple layers of cells and small concentric luminal spaces. Tumors with solid tumor nests containing glandular lumina may display CEACAM5 apical membrane staining of the lumina. In the case of luminal staining at ≥2+ intensity, it is hypothesized that all cells within the nest may have membrane association to the lumen, and therefore all cells within the nest should be considered positive (FIG. 10 and FIG. 11).
Alveolar pattern: Gland-like structures (single or multi-layered) containing irregularly shaped luminal spaces of different sizes. Cells exhibiting apical membrane staining at ≥2+ intensity in the luminal surface are considered positive. Cell layers behind those luminal facing cells may also have membrane associated to the positive lumen and are therefore considered similarly positive. More details and examples are provided in Appendix FIG. 3 and FIG. 12.

Cells with Signet Ring Morphology: Cells with signet ring cell morphology can exhibit high intensity staining in the intravacuolar space and cytoplasm. Membrane staining in these cases may be difficult to differentiate due to the cytoplasmic staining intensity. For cells with signet ring cell morphology, the high intensity staining tumor cells are considered positive for membrane staining and are scored relative to the cytoplasm intensity. For example, if the cytoplasm intensity is ≥2+ in a cell with signet ring morphology, then the cell is considered positive for CEACAM5 (FIG. 13).

High Cytoplasmic Staining Cells: Cytoplasmic staining without unequivocal membrane staining is generally not considered as positive staining. However, positivity may be inferred for high cytoplasmic staining tumor cells (3+ intensity) when positive membrane staining is identified in tumor cells that are within a 10× objective field of the high cytoplasmic staining cells (FIG. 4).

Viable Intraluminal Cells: Viable tumor cells within intraluminal and alveolar spaces should be included in scoring if membrane staining is ≥2+ intensity and if viability can be confirmed. A matched H&E slide may be needed to determine viability. Care must be taken to distinguish tumor cells from macrophages, which should not be scored. If cells are non-viable, or if viability or tumor identity cannot be confirmed, intraluminal cells should be excluded (FIG. 14).

Intracytoplasmic Lumina: Cells with intracytoplasmic lumina with luminal staining at ≥2+ intensity are considered positive (FIG. 15). The H&E may be used to determine whether a specimen contains this expression pattern.

### Non-Specific Background Staining:

Non-specific background staining for NCR and CEACAM5 stained slides should be of ≤1+ average intensity across the specimen.

The following are excluded from evaluation of non-specific background staining:
Stroma that is near high intensity stained tumor cells may have some staining. Stromal staining is not scored as non-specific background staining (FIG. 7).
Necrosis can stain at high intensity and is not scored as non-specific background staining (FIG. 6).
Mucin can stain at high intensity and is not scored as non-specific background staining (FIG. 16).

Non-specific background staining is assessed across the entire specimen, not focally (i.e., in an area that amounts to a small proportion of the total area). Focal staining at >1+ intensity is acceptable if counterbalanced by areas of no staining such that the average intensity across the entire specimen is ≤1+.

### System Controls

Before examining the IHC stained tumor specimen(s), confirm that the IHC staining of control tissues is satisfactory.

A positive control tissue and a negative control tissue are run in parallel with patient tissues. The NCR staining is run on control tissues in order to confirm the absence of non-specific staining for the control tissues within that staining procedure. See FIG. 17 for a representative image of a Positive Control Tissue and FIG. 18 for a representative image of a Negative Control Tissue.

### Control Tumor Tissues:

Control tumor tissues should be identified from pre-screened non-squamous NSCLC tissues. Controls should be fresh biopsy/surgical non-squamous NSCLC specimens fixed, processed and embedded as soon as possible in the same manner as the test specimen(s).

### Negative Control Reagent (NCR)

Negative Control Reagent contains a species matched antibody non-specific to CEACAM5. Control tissues stained with NCR must exhibit no specific membrane staining, and the presence of non-specific background staining should be a score of ≤1+ average intensity across the entire specimen. If specific staining occurs in the NCR stained tissues, then results are considered invalid, the checkbox for “Fail” should be recorded on the scoring form, and the staining should be repeated on the controls and test specimens.

### Positive Control Tissues

Positive control tissues are evaluated to ensure tissue preparation and satisfactory staining techniques. One positive control tissue is included in each staining procedure.
Tissues selected for use as the positive tissue controls should give moderately positive staining (≥2+ intensity) to detect subtle changes in assay sensitivity.
Non-specific background staining should be of ≤1+ average intensity across the specimen (see Section 2.2).
Specimens processed differently from the test sample(s) validate reagent performance only and do not verify tissue preparation.
Known positive tissue controls should only be utilized for monitoring the correct performance of processed tissues and test reagents, NOT as an aid in formulating a specific diagnosis of patient samples.

### Negative Control Tissues

Non-squamous NSCLC negative control tissues are evaluated to ensure tissue preparation, satisfactory staining techniques, verify the specificity of the primary antibody, and to provide an indication of non-specific staining.
Different cell types present in most tissue sections offer internal negative control sites (this should be verified by the user).
A suggested negative control tumor tissue for non-squamous NSCLC is one that shows no membrane staining in tumor cells.
Non-specific background staining should be of ≤1+ average intensity across the specimen.
Known negative tissue controls should only be utilized for monitoring the correct performance of processed tissues and test reagents, NOT as an aid in formulating a specific diagnosis of patient samples.

### Acceptance Criteria for Control Specimens

Based on the staining results of the control specimens, accept or reject the results of the test specimen according to the following criteria:
Accept the test specimen results if controls meet all requirements.
If control tissue slides do not meet requirements, then test specimen results should be considered invalid and the staining should be repeated, including tissue controls and tissue samples.
If Negative Control Reagent (NCR) staining results do not meet requirements, then test specimen results should be considered invalid and the staining should be repeated, including tissue controls and tissue samples.

Once proper staining of the control materials has been established, the staining is judged to be satisfactory for assessment of test samples.

Patient Tissue Slide Stained with NCR

Negative Control Reagent is used in place of the CEACAM5 specific primary antibody and aids in interpretation of specific staining at the antigen site.

Absence of membrane staining verifies the specific labeling of the target antigen by the CEACAM5 primary antibody on the patient specimen. Staining by the NCR must not show any membrane staining at any intensity. Non-specific background staining should be ≤1+ average intensity across the specimen.

### Slide Reading Order and Evaluation

For each staining run, slides should be examined in the order presented in the table below to determine the validity of the staining run and enable assessment of stained sample tissue.

| Specimens | Rationale | Requirements |
|---|---|---|
| 1. Patient | A hematoxylin and eosin (H&E) | The H&E and CEACAM5 antibody stain |
| tissue stained | stain of the patient tissue is | should be performed on serial sections from |
| with H&E | evaluated first to assess tissue | the same paraffin block of the specimen. A |
| | histology and preservation | minimum of 100 viable tumor cells must be |
| | quality. | present for the specimen to be considered |
| | | adequate for CEACAM5 evaluation. |
| | | Tissue specimens should be undamaged, |
| | | well-preserved, and should confirm tumor |
| | | indication. |
| 2. Positive | The positive control tissue | Controls should be biopsy/surgical |
| control tissue | stained with CEACAM5 | specimens, fixed, processed and embedded |
| stained with | primary antibody should be | as soon as possible in the same manner as |
| CEACAM5 | examined next. Known positive | the patient tissue(s). |
| primary | control tissue should only be | Positive control tissue should be included in |
| antibody | utilized for monitoring the | each staining procedure. On-slide tissue |
| (Lab-supplied) | correct performance of | controls are recommended |
| | processed tissues and test | Positive control tissue should be: non- |
| | reagents, NOT as an aid in | squamous NSCLC specimen previously |
| | formulating a specific diagnosis | screened by the laboratory and known to |
| | of patient tissue. | express CEACAM5. Tumor cells should |
| | | exhibit membrane staining at moderate |
| | | intensity. |
| | | Non-specific background staining should |
| | | be ≤1 + average staining intensity across |
| | | the specimen. |
| | | If the positive control tissue fails to |
| | | demonstrate appropriate positive staining, |
| | | then results of the test specimen (patient |
| | | tissue) should be considered invalid. |
| 3. Positive | Negative Control Reagent may | Tissue stained with NCR must exhibit no |
| control tissue | be used to stain the positive | membrane staining. |
| stained with | control tissue specimen if | Non-specific background staining should |
| Negative | needed for troubleshooting | be ≤1 + average staining intensity across |
| Control | purposes. | the specimen. |
| Reagent | | |
| (NCR) | | |
| (Lab-supplied) | | |
| 4. Negative | The negative control tissue | Controls should be biopsy/surgical |
| control tissue | stained with CEACAM5 | specimens, fixed, processed and embedded |
| stained with | primary antibody should be | as soon as possible in the same manner as |
| CEACAM5 | examined next to verify the | the patient tissue(s). |
| primary | labeling specificity of the target | Negative control tissue should be included |
| antibody | antigen by the primary antibody. | in each staining procedure. On-slide tissue |
| (Lab-supplied) | | controls are recommended. |
| | | Negative control tissue should be: non- |
| | | squamous NSCLC specimen previously |
| | | screened by the laboratory and known to |
| | | not express CEACAM5 staining. Tumor |
| | | cells should exhibit no membrane staining. |
| | | Non-specific background staining should |
| | | be ≤1 + average staining intensity across |
| | | the specimen. |
| | | If the negative control tissues fail to meet |
| | | the above specifications, the results of the |
| | | patient tissue should be considered invalid. |
| 5. Negative | Negative Control Reagent may | Tissue stained with NCR must exhibit no |
| control tissue | be used to stain the negative | membrane staining in tumor cells. |
| stained with | control tissue specimen if | Non-specific background staining should |
| Negative | needed for troubleshooting | be ≤1 + average staining intensity across |
| Control | purposes. | the specimen. |
| Reagent | | |
| (NCR) | | |
| (Lab-supplied) | | |
| 6. Patient | Examine patient tissue stained | Patient tissue stained with NCR must |
| tissue stained | with the Negative Control | exhibit no membrane staining in tumor |
| using the | Reagent. Negative Control | cells. |
| Negative | Reagent is used in place of the | Non-specific staining should be ≤1 + |
| Control | primary antibody and aids in | average staining intensity across the |
| Reagent | interpretation of specific | specimen. |
| (NCR) | staining at the antigen site. | If patient tissue stained with NCR fails to |
| | | demonstrate appropriate staining, the |
| | | corresponding patient tissue stained with |
| | | primary antibody is considered non- |
| | | evaluable and the patient tissue must be |
| | | retested. |
| 7. Patient | Examine the patient tissue | All viable tumor cells on the entire |
| tissue stained | stained with the CEACAM5 | CEACAM5-stained slide must be evaluated |
| using the | antibody last to assess | for CEACAM5 and included in the scoring |
| CEACAM5 | CEACAM5 protein status. | assessment. A minimum of 100 viable |
| primary | | tumor cells must be present for the |
| antibody | | specimen to be considered adequate for |
| | | CEACAM5 evaluation. |
| | | Positive staining intensity should be |
| | | assessed within the context of any non- |
| | | specific staining observed in the staining |
| | | procedure. NCR is recommended for this |
| | | assessment if non-specific staining is |
| | | observed. |
| | | As with any immunohistochemical test, a |
| | | result showing no staining means that the |
| | | antigen was not detected, not necessarily |
| | | that the antigen was absent in the |
| | | cells/tissue assayed. |
| | | Refer to Scoring Guidelines for details on |
| | | patient tissue evaluation. |

NCR Stained Specimen

The Negative Control Reagent (NCR) should be examined with a section of each patient specimen to evaluate non-specific staining and allow interpretation of specific staining. FIG. 19 illustrates the evaluation of the H&E, then the NCR, and finally the CEACAM5 IHC slide for non-squamous NSCLC.

Acceptance Criteria:
Staining by NCR must not exhibit any specific membrane staining at any intensity.
Non-specific background staining should be ≤1+ average intensity across the specimen.

### CEACAM5 Stained Specimen

Carefully examine the entire tumor specimen and determine the percentage of CEACAM5 positive tumor cells at ≥2+ intensity:

$$\% CEACAM 5 positive = 100 \times \frac{\begin{matrix}\# of tumor cells expressing CEACAM 5 \\ membrane staining at \geq 2 + intensity\end{matrix}}{Total \# of viable tumor cells present in the section}$$

Examples of staining at the individual intensities are shown in FIG. 8. The estimated number of total viable tumor cells in the entire tissue section will be used as the denominator for computation of the score.

The CEACAM5 diagnostic status (positive/negative) of non-squamous NSCLC specimens is determined by the percentage of viable tumor cells expressing CEACAM5 positive (≥2+ intensity) membrane staining.
Tumor cells are CEACAM5 positive if they exhibit either partial or complete circumferential membrane staining at 2+ and 3+ intensity, see FIG. 3.
CEACAM5 staining may appear as punctate or linear membrane staining as illustrated in FIG. 2.
Review CEACAM5 expression patterns for important scoring details.
Non-specific background staining should be ≤1+ average intensity across the specimen.

For evaluation of the CEACAM5 immunohistochemical staining and scoring, 4× objective magnification can be used for initial assessment of the entire specimen followed by scoring tumor cell staining at ≥2+ intensity with the 4× to 10× objectives. 20× may be used to identify and/or confirm tumor cells exhibiting 1+ and punctate and/or delicate 2+ membrane staining as well as 2+ intracytoplasmic lumina. The 20×-40× objectives can also be utilized to confirm membrane localization of staining, if needed. The 40× objective should not be routinely used for scoring (i.e., to assess intensity and percent positivity).

Conduct scoring of CEACAM5 positivity with the following resolution: in the range from zero to 10%, score positivity in 1% increments, except when in the range from zero to 1%, designate “<1%”. In the range from 10% to 100%, positivity may be scored in 5% increments, but smaller increments may be used if necessary.

Elements to Exclude from Scoring and from Evaluation of Non-Specific Background

Additional tissue elements that may be present and may show apparently genuine CEACAM5 staining include:
Mucin (see FIG. 16)
Sloughed intraluminal non-viable cells
Benign epithelial cells (see FIG. 5)
Immune cells (e.g., macrophages; see FIG. 14).

Staining of these areas are excluded from determining CEACAM5 expression status.

Examples of endogenous pigments that appear in some specimens, and how they can be differentiated from genuine CEACAM5 staining, are shown in FIG. 9.

The CEACAM5 positivity status is determined based on pre-determined cut-off point of CEACAM5 positive staining at ≥2+ intensity level and recorded.

The term “indeterminate” is utilized to denote a sample that has met the criteria for evaluation, but scoring of tumor cell membrane staining has been hampered for reasons attributable to the biology of the tumor tissue sample rather than because of improper sample preparation or handling.

Staining may also occur in certain regions of a tissue specimen such as the stroma, necrotic areas, and edge artifact in peripheral regions of the tissue. Stromal staining may occur in specimens exhibiting highly positive CEACAM5 expression, as shown in FIG. 7. Staining of these tissue elements are excluded from determining CEACAM5 expression status and from the evaluation of non-specific background staining (see Section 2.2). Examples of some of these staining artifacts are shown in FIG. 6 and FIG. 7.

FIG. 1 illustrates a representative image of CEACAM5 staining in non-squamous NSCLC using a CEACAM5 IHC (antibody clone) 769 assay, as described herein.

FIG. 2 illustrates a representative image of positive staining examples: Punctate (red arrow, or arrow at left edge image) and linear (black arrow, or arrow at middle of image) membrane staining at greater than ≥2+ intensity are considered positive; the 40× image is shown here for detailed illustration, however scoring and calculation of % CEACAM5 positive membrane staining also can be measured at the 4× and/or 10× objectives.

FIG. 3 of partial and complete membrane staining: arrow “a” indicates complete membrane staining; arrow “b” indicates partial, basolateral and apical (luminal) membrane staining; and arrow “c” indicates partial, apical (luminal) membrane staining (alveolar pattern).

FIG. 4 illustrates CEACAM5 scoring, as provided herein: cytoplasmic staining is generally not considered for CEACAM5 scoring, and only membrane staining is relevant for calculating the CEACAM5 percent positivity score. However, positivity may be inferred for cells with high cytoplasmic staining when positive membrane staining is identified in tumor cells that are within a 10× objective field of the high cytoplasmic staining cells. In this case, red (or longer) arrows indicate areas where high cytoplasmic staining without distinguishable membrane staining is observed. For these areas, the cells are scored as positive for CEACAM5 because tumor cells within a 10× objective field (black (or shorter) arrows) have discernible membrane staining and indicate that the area is highly positive for CEACAM5.

FIG. 5 illustrates an exemplary benign tissue staining example: staining of normal (non-neoplastic) tissue, as shown here, is excluded from CEACAM5 scoring protocols as provided herein.

FIG. 6 illustrates an image demonstrating that staining may occur in necrotic tissue and at a range of intensities. Necrotic regions should be excluded from scoring using CEACAM5 scoring protocols as provided herein, and not considered as non-specific background staining.

FIG. 7 illustrates an image demonstrating that only tumor cells (black (or longer) arrow) are scored for CEACAM5 positivity, and any surrounding stroma (red (shorter) arrows) showing CEACAM5 staining is excluded from the percent score of CEACAM5 staining protocols as provided herein, and not considered as non-specific background staining.

FIG. 8 illustrates images of 1+ (upper row), 2+ (middle row), 3+ (lower row) staining intensities using CEACAM5 scoring protocols as provided herein. The 2+ and 3+ membrane staining intensities are considered CEACAM5 positive according to the CEACAM5 scoring protocols as provided herein.

FIG. 9A-B illustrate images demonstrating that pigments that may be present in specimens, for example, pigments such as anthracotic pigment (FIG. 9A arrow) and hemosiderin (FIG. 9B arrow) are distinguishable from DAB precipitate at moderate to high magnification and should not be included in scoring for CEACAM5 positivity using CEACAM5 scoring protocols as provided herein.

FIG. 10A-B illustrate images of luminal staining details and nested patterns: FIG. 10A show H&E (hematoxylin and eosin) staining at 20×; and FIG. 10B shows CEACAM5 positive apical membrane staining at 20× in the luminal portion of the nest. When a nest has layers of tumor cells and the lumen is positive, then all the tumor cells within the nest are positive. In this case, using CEACAM5 scoring protocols as provided herein, the cells within the dashed region are considered positive.

FIG. 11 illustrates an image of luminal staining details and nested patterns: the nested expression pattern is solid tumor nests delimited by stroma containing multiple layers of cells and small concentric luminal spaces. If a nest displays positive luminal staining, then all cells within the nest are considered positive using CEACAM5 scoring protocols as provided herein. Arrows indicate nests that are positive or negative for CEACAM5.

FIG. 12A-B illustrate images of luminal staining details and alveolar patterns: When a structure with this pattern has multiple layers of cells and the lumen is positive, cells exhibiting apical membrane staining at greater than or equal to (≥) 2+ intensity in the luminal surface are considered positive and cell layers behind those luminal facing cells are considered similarly positive using CEACAM5 scoring protocols as provided herein. FIG. 12A: all cells in the outlined alveolar structure are positive; and FIG. 12B: the alveolar structure at the center of this panel contains positive and negative cells.

FIG. 13A-B illustrate images of H&E staining (FIG. 13A) and CEACAM5 staining (FIG. 13B) of cells with signet ring morphology. Cells with signet ring morphology can exhibit high intensity staining in the intravacuolar space and cytoplasm. Membrane staining may be difficult to differentiate due to the cytoplasmic staining intensity. In these cases, the tumor cells are considered positive for membrane staining and are scored relative to the cytoplasm intensity, i.e., if the cytoplasm intensity is ≥2+ in the signet ring subtype, then the cell is considered positive using CEACAM5 scoring protocols as provided herein.

FIG. 14A-D illustrate images of viable tumor cells in intraluminal spaces (FIG. 14A; FIG. 14B) should be scored using CEACAM5 scoring protocols as provided herein if viability can be confirmed: FIG. 14A is H&E staining of tumor cells, and FIG. 14B is CEACAM5 staining of tumor cells, as described herein. An H&E may be needed for this assessment. Care should be taken to distinguish tumor cells from macrophages FIG. 14C-D: Macrophages and non-viable tumor cells should not be scored, FIG. 14C is H&E staining, and FIG. 14D is CEACAM5 staining, as described herein.

FIG. 15 illustrates images of intracytoplasmic lumina (arrows) staining demonstrating that CEACAM5 may be present. H&E staining may be used to confirm the presence of intracytoplasmic lumina. These cells are considered positive when staining is ≥2+ and should be included in scoring using CEACAM5 scoring protocols as provided herein.

FIG. 16A-B illustrate images demonstrating that mucin can stain at high intensity (H&E staining FIG. 16A, and CEACAM5 staining FIG. 16B); this is not considered as non-specific background staining, and it is also excluded from CEACAM5 percent score using CEACAM5 scoring protocols as provided herein. Example mucin staining shown in boxed area.

FIG. 17A-B illustrate positive control tissue images: non-squamous NSCLC tissue at 10× (FIG. 17A) and 20× (FIG. 17B) demonstrating a range of weak (1+, red arrows, or top two arrows at center of image and second arrow down from top right of image) to moderate (2+, black arrows, or two lower arrows at center and bottom right of image and arrow at top right of image) intensity membrane staining using CEACAM5 scoring protocols as provided herein.

FIG. 18A-D illustrate negative control tissue images: FIG. 18A: H&E stain of non-squamous NSCLC; FIG. 18B: a low magnification objective can be used to initially scan tissue and observe any CEACAM5 staining; FIG. 18C: a higher magnification of 10× can be used to confirm if there is any staining; and, FIG. 18D: a 20× objective can be used to visualize if there is any specific membrane staining at the 1+ intensity. In this example, there is no CEACAM5 staining and this specimen can serve as a good negative control tissue.

FIG. 19, top panel, illustrates an image of an H&E (hematoxylin and eosin) stain of a tissue specimen, which is evaluated first by H&E to assess tissue histology and preservation quality;

FIG. 19, middle panel, illustrates a negative control reagent slide, this is used to evaluate non-specific staining and allow interpretation of a CEACAM5 stained slide; and

FIG. 19, lower panel, illustrates CEACAM5 staining, which is assessed and, using CEACAM5 scoring protocols as provided herein, is considered positive staining if it is complete and/or partial plasma membrane staining at greater than or equal to (≥) 2+ intensity.

### Example 2: External Reproducibility Study of CEACAM5 IHC 769 on Non-Squamous Non-Small Cell Lung Cancer Specimens

This example demonstrates use of CEACAM5 staining protocols as provided herein.

The objective of this study was to evaluate:
the investigational device, CEACAM5 IHC 769, with respect to Inter-Site, Intra-Site/Inter-Day, Inter-Observer and Intra-Observer reproducibility on a set of formalin-fixed, paraffin-embedded (FFPE) human non-squamous non-small cell lung cancer tissue specimens performed at three external test sites; and,
the inter-site, intra-site/inter-day, inter-observer, and intra-observer reproducibility of CEACAM5 IHC 769 using the Dako Omnis instrument on FFPE non-squamous NSCLC specimens at the ≥2+ intensity ≥50% CEACAM5 positive tumor cells. The results of this study is be used to demonstrate the reproducibility of the assay per applicable regulatory guidelines.

CEACAM5 IHC 769 is a qualitative immunohistochemical (IHC) assay using Monoclonal Mouse Anti-CEACAM5 Clone 769, intended for use in the detection of human CEACAM5 protein in formalin-fixed, paraffin-embedded (FFPE) non-squamous non-small cell lung cancer.

CEACAM5 IHC 769 assay is used as an aid to identify patients that may benefit from participation in clinical trials with Sanofi SAR408701 (Tusamitamab ravtansine) under development as a treatment for patients with advanced non-squamous NSCLC.

Anti-CEACAM5 antibody-drug conjugate Tusamitamab ravtansine is an immunoconjugate consisting of anti-carcinoembryonic antigen-related cell adhesion molecule 5 (CEACAM5) conjugated to a cytotoxic agent, with potential antineoplastic activity. Upon administration of anti-CEACAM5 antibody-drug conjugate Tusamitamab ravtansine, the antibody moiety targets CEACAM5 on tumor cells. Upon antibody/antigen binding and internalization, the immunoconjugate releases the cytotoxic agent, which results in tumor cell death.

The purpose of this study was to evaluate the performance of CEACAM5 IHC 769 with regard to inter-site, intra-site/inter-day and inter-observer, intra-observer reproducibility examined at assay cut-off of ≥2+ intensity ≥50% CEACAM5 positive tumor cells.

The diagnostic endpoint was defined as positive if the percent of CEACAM5 expressing tumor cells at ≥2+ intensity is ≥50%. Negative is defined as ≥2+ intensity staining in <50% staining of tumor cells. This study assessed the reproducibility of the diagnostic endpoint (positive/negative) evaluation using CEACAM5 IHC 769 according to the CEACAM5 staining protocol as provided herein.

Endpoints: the following endpoints of the study were assessed:
Inter-Site reproducibility across three sites and five Rounds
Intra-Site/Inter-Day reproducibility across five Rounds within three sites
Inter-Observer reproducibility across three pathologists and three Reads
Intra-Observer reproducibility across three Reads by each of three pathologists.

This study was a three-site, blinded, randomized reproducibility study of CEACAM5 IHC 769 on FFPE human non-squamous non-small cell lung cancer (nsNSCLC) tissue specimens.

The study was divided into two parts: Part A (inter-site and intra-site/inter-day endpoints) and Part B (inter-observer and intra-observer endpoints). All slides were re-randomized for evaluation order between Rounds/Reads and interpreted by the pathologists blinded from specimen identity as well as the previous evaluations. The slide sets were read by site pathologists trained on the scoring algorithm The maximum washout period was not to exceed 30 days; if it had been exceeded, a proficiency test and/or refresher training was given.

Study Part A: Each of the three external sites performed automated staining runs, testing 50 FFPE human nsNSCLC specimens. The specimens were qualified at Agilent and distributed to the three sites with each site receiving five replicate sets. Staining was performed on five non-consecutive days, over a period of at least twenty days.

Study Part B: One set of 66 nsNSCLC pre-stained specimens was qualified at Agilent and rotated across the three testing sites to be evaluated three times by the same pathologist at each site. Included in the set, but not analyzed, were four unique wildcards per read.

Efforts were made to select specimens representing the full range of biomarker expression with a balanced distribution of positive and negative samples based on the cut-off of ≥2+ intensity in ≥50% CEACAM5 positive tumor cells. Approximately 20% of specimens were selected in the near cut-off range of ≥2+ intensity from ≥40% to ≤60% CEACAM5 positive tumor cells.

Study Part A: Targeted enrollment included 50 specimens, consisting of approximately 25 positive and 25 negative samples.

Study Part B: A set of 66 specimens, consisting of approximately 33 positive and 33 negative samples for analysis. Additionally, 4 wildcard specimens for each read (a total of 12) were included to mitigate the possibility of recall bias.

### Statistical Analysis: Agreement Estimates for all Study Endpoints:

The analytical agreement of the diagnostic outcome (positive/negative) were evaluated for Study Part A (Inter-Site and Intra-Site) and Study Part B (Inter-Observer and Intra-Observer) using the following methods:

1. Evaluation of NPA, PPA and OA, based on consensus score, with Bootstrap confidence intervals.

Negative percent agreement (NPA), positive percent agreement (PPA), and overall agreement (OA) were calculated for inter-site, intra-site/inter-day, inter-observer, and intra-observer reproducibility, by making comparisons of diagnostic status (positive/negative) from each slide to the consensus diagnostic outcome. The two-sided 95% confidence intervals on NPA, PPA and OA were computed using the Bootstrap method.

2. Analysis on IHC Staining Percent Scores.

The variability in CEACAM5 staining percent scores (continuous data) were assessed by analysis of variance (ANOVA) based on a mixed model with Site and Round (Study Part A), and Observer and Read (Study Part B) as random factors. Mean, standard deviation and coefficient of variation for the site and the observer measures were reported for each specimen.

Acceptance Criteria: The lower bound of a two-sided 95% confidence interval for NPA, PPA, and OA must be at least 85% for each reproducibility measure.

Results: The results of this external reproducibility study demonstrate high concordance of all diagnostic endpoints and met all pre-determined acceptance criteria for each of the study endpoints at the ≥2+ Intensity ≥50% CEACAM5 Positive Tumor Cells Cut-off.

Conclusions: The results of this study demonstrate that concordance of diagnostic endpoint decision met pre-determined acceptance criteria. The reproducibility results for a ≥2+ Intensity ≥50% cut-off for CEACAM5 positive and negative status across three observers met acceptance criteria of a CI lower bound ≥85% for NPA, PPA, and OA reproducibility estimate.

### Overall Study Design

This study was designed to assess the assay performance of CEACAM5 IHC 769 in three independent CLIA-certified clinical laboratories. The study utilized blinded and randomized FFPE tissue sections from non-squamous NSCLC specimens which express differing levels of CEACAM5. All specimens in the study were evaluated by pathologists certified in the scoring of CEACAM5. The study consisted of a Part A, that assessed the combined staining and scoring reproducibility, while Part B assessed observer evaluation reproducibility.

The study was designed to investigate the reproducibility of positive/negative interpretation of CEACAM5 IHC 769 based on: 2+ intensity and 50% CEACAM5 positive tumor cells score as the pre-determined cut-off value, with ≥2+ intensity ≥50% defined as positive and ≥2+ intensity <50% defined as negative.

The distribution of the samples for Part A and Part B were based on pre-defined % CEACAM5 positive tumor score range for negative, positive, and near cut-off samples and is described in Table 1:

TABLE 1
CEACAM5 Score Range for Pre-Defined Specimen Categories

| Specimen | |
|---|---|
| Category | CEACAM5 Score |
| Negative | <50% CEACAM5 positive tumor cells at ≥2 + intensity |
| Near Cut-off | 40% to 60% CEACAM5 positive tumor cells at ≥2 + |
| | intensity |
| Positive | ≥50% CEACAM5 positive tumor cells at ≥2 + intensity |

Approximately 20% of specimens were selected in the near cut-off range.

For Part A: Staining was performed over a period of at least twenty days for each site. Each set was evaluated by a qualified pathologist, who was provided with H&E, CEACAM5, and NCR slides for each case. Each evaluation session was limited to a maximum of 30 specimens scored per day to minimize the potential for scoring fatigue. There was a minimum of 14 calendar days washout period between each of the 3 reads per observer for each specimen, but no longer than 30 days, built into the study. Schedules were coordinated with lab personnel to ensure study requirements were satisfied.

For Part B: Inter-Observer, Intra-Observer Reproducibility

This portion of the study was conducted using a single set of 66 permanently mounted CEACAM5, NCR, and H&E stained slides prepared at Agilent and provided to the study sites (rotated to be evaluated three times by each site), ensuring that the scores (to be analyzed) from each pathologist were based on the same set of slides. To mitigate recall bias, a set of 4 wildcard specimens (each set unique to each Read) were added and evaluated, but not included in the data analysis. After all sites went through a round of evaluation, assigned Agilent personnel incorporated the next unique wildcard set and randomized the slide set read order according to the randomization procedure. There was a minimum of 14 calendar days washout period between each of the 3 reads per observer for each specimen, but no longer than 30 days, built into the study.

Methods to Avoid Bias:
The specimens within an evaluation were completely anonymized and specimen ID blinded from the testing sites (apart from personnel assigned to randomize H&E specimens).
All IHC stained slides were randomized based on order of evaluation and the identification of the specimens were blinded to the pathologist.
The pathologists were instructed to not discuss the scoring results of the specimens with each other.
The five inter-day tests (Rounds) were performed for Part A on non-consecutive five days.
A 14 to 30-day washout period was followed between each of the five scoring evaluations (Rounds) for part A and between each of the three scoring evaluations (Reads) for Part B at each site.
For Part B, four pre-determined wildcard specimens were randomized into the testing specimens with four different wildcards for each of the three reads.

### Materials

### Description of Investigational Diagnostic Medical Device

CEACAM5 IHC 769 contains optimized reagents and a staining protocol required to complete an immunohistochemical (IHC) staining procedure for FFPE specimens using the Dako Omnis. One kit includes reagents sufficient to perform 60 tests (D60362). PGP-27 Tl

TABLE 2
CEACAM5 IHC 769 Components (1 Assay Kit)

| | | Quantity × |
|---|---|---|
| Component | Component description | Volume |
| Primary Antibody: | Monoclonal mouse Anti- | 1 × 12 mL |
| Monoclonal Mouse Anti- | CEACAM5 in a buffered solution, | |
| Human CEACAM5, | containing stabilizing protein, | |
| Clone 769 | and 0.015 mol/L sodium azide | |
| Negative Control | Monoclonal rabbit control lgG | 1 × 12 mL |
| Reagent (NCR) | antibody in a buffered solution, | |
| | containing stabilizing protein, and | |
| | 0.015 mol/L sodium azide | |

Only one lot number of the CEACAM5 IHC 769 kit was used by the sites in Part A.

### Device Operation

CEACAM5 IHC 769 contains optimized reagents and the protocol required to complete an IHC staining procedure of FFPE specimens using the Dako Omnis. Following incubation with the Primary Monoclonal Antibody to CEACAM5 or the Negative Control Reagent (NCR), specimens are incubated with a ready-to-use visualization reagent consisting of secondary antibody molecules and horseradish peroxidase molecules coupled to a dextran polymer backbone. The enzymatic conversion of the subsequently added chromogen results in the precipitation of a visible reaction product at the site of antigen. The specimen may then be counterstained and mounted. Results are read using a light microscope. CEACAM5 IHC 769 (product code GE025) is applicable for automated staining using the Dako Omnis instrument.

The working procedures followed the Instructions for Use (IFU) for CEACAM5 IHC 769 for nsNSCLC specimens (D60362).

The interpretation of the staining followed the scoring guidelines in the IFU (D60362) and detailed scoring methods in the Pathology Scoring Manual for CEACAM5 IHC 769 for nsNSCLC specimens (D55521) CEACAM5-positive staining is defined as any partial or complete tumor cell plasma membrane staining at ≥2+ intensity.

Screening of tissues for the study were performed using CEACAM5 IHC 769 according to IFU (D60362) of the device. A certified pathologist confirmed the selected specimens met the tissue inclusion/exclusion criteria prior to start of study. Screening of specimens were performed by certified readers (pathologist or scientist).

### Specimen Profile Set Design (Part a and Part B)

The tissue specimens were selected according to the study protocol and External Specimen Selection and Vetting guidance. The summarized profile of nsNSCLC specimens selected for this study, expressed in number of specimens (n) and percent of total set (%) is shown in Table 3:

TABLE 3
Summarized Profile of the nsNSCLC
Specimen set for Part A and Part B

| | | |
|---|---|---|
| Specimen | Part A (n = 50) | Part B (n = 66) |

| Definition | n | % | N | % |
|---|---|---|---|---|
| Positive | 25 | 50.0 | 33 | 50.0 |
| Negative | 25 | 50.0 | 33 | 50.0 |
| NCO | 11 | 22.0 | 13 | 19.7 |

Scoring of nsNSCLC Specimens

Scoring was performed according to IFU of the device as well as the pathology scoring manual, using a bright field microscope. A brief overview of the scoring process is described herein.

This reproducibility study employs the cut-off of ≥50% CEACAM5 positive tumor cells at ≥2+ staining intensity on tumor cell membrane. Specimens were classified as positive (≥2 cut-off) or negative (<cut-off) based on this cut-off.

The entire specimen was evaluated. All viable tumor cells on the entire CEACAM5 stained slide were evaluated and included in the CEACAM5 scoring assessment. The H&E stained slide was used to determine whether a sufficient number of tumor cells were present in the tissue section of the given specimen. A minimum of 100 viable tumor cells must be present in the CEACAM5 stained slide to determine the percentage of CEACAM5 stained cells. Positivity was defined as any partial or complete tumor cell plasma membrane staining at greater than or equal to 2+ intensity (≥2+).

### Statistical Analysis

Part A of the study utilized 50 specimens for analysis and Part B utilized 66 specimens for analysis. Specimens were selected so that approximately 50% of the total count would be negative, and 50% would be positive, based on diagnostic outcome from specimen screening. Efforts were made to enrich the specimen set with approximately 20-25% in the near cut-off range. The specimen sets for Study Part A and Study Part B contained overlapping specimens.

Specimens near the cut-off were selected based on the percent positive scores generated during the vetting and selection process as those within the near cut-off range of 40% to 60% at an intensity of 2+ or greater. Specimens reported to be near cut-off at study conclusion were based on whether the median of the continuous scores of the study pathologists was within the near cut-off range.

### Calculation of Percent Agreements and Confidence Intervals

For the reproducibility measures of inter-site and intra-site (inter-day), and inter-observer and intra-observer, the agreement estimates, and respective confidence intervals were calculated by the following method: comparison to consensus to calculate NPA/PPA/OA.

Calculating percent agreements NPA and PPA required comparisons to a reference condition. For reproducibility parameters such as site, day, observer and read, no reference exists. Comparisons were made on the IHC diagnostic status (positive/negative) between each test condition and the consensus outcome (most frequently occurring observation). The total number of comparisons per specimen was equal to the total number of scores.

Results

### Reported Results for Part a and Part B

The following section presents the inter-site, intra-site reproducibility, inter-observer and intra-observer reproducibility statistical analysis results. All line data (i.e. recorded data points of continuous CEACAM5 and data points of negative/positive diagnostic endpoints based on the ≥2+ Intensity ≥50% cut-off for Part A and Part B) are tabulated.
The following results are reported in the statistical analysis summary tables (Tables 5-8):
Specimen distribution profile: number (n) and percent (%) of specimens evaluated as CEACAM5 positive (P) or negative (N) and near the ≥2+ Intensity ≥50% cut-off (NCO 50%); a specimen is described as “near the 50% cut-off” if the median value of its CEACAM5 scores falls within 40% to 60%
Number of comparisons to consensus: number of concordant negative, concordant positive, discordant negative, and discordant positive comparisons
NPA
PPA
OA
95% confidence interval for each NPA, PPA, and OA

### Overall Study Results

Table 4 provides an overall summary of the specimen profile distribution based on the external reproducibility pathologist's evaluation:

TABLE 4
Summarized Profile of nsNSCLC Specimens at the ≥2 +
Intensity ≥ 50% Cut-off Based on Median of External
Pathologists' Raw Scores for each Specimen

| | | |
|---|---|---|
| Specimen | Part A (n = 50) | Part B (n = 66) |

| Definition | n | % | N | % |
|---|---|---|---|---|
| Positive | 25 | 50.0 | 34 | 51.5% |
| Negative | 25 | 50.0 | 32 | 48.5% |
| NCO | 12 | 24.0 | 11 | 16.7% |

TABLE 5
Inter-Site Reproducibility Analysis, ≥2 + Intensity ≥ 50% Cut-off
nsNSCLC, Inter-Site, ≥50 Cutoff
Specimen Distribution Summary

| Definition | Score Range | # Blocks | % Blocks |
|---|---|---|---|
| Negative | <50 | 25/50 | 50.0% |
| Positive | ≥50 | 25/50 | 50.0% |
| Total Near Cutoff | 40-60 | 12/50 | 24.% |

Comparisons to Consensus Summary

| | |
|---|---|
| Negative Consensus | Positive Consensus |

| Concordant | Discordant | Concordant | Discordant | |
|---|---|---|---|---|
| Negatives | Positives | Positives | Negatives | Total |
| 354 | 21 | 363 | 21 | 750 |

Agreement Summary

| | | 95% Confidence Interval | |
|---|---|---|---|
| Performance | Point | (Bootstrap) | |
| Criteria | Estimate | Lower-bound 2.5% | Upper-bound 97.5 |
| NPA | 94.4% | 90.1% | 97.9% |
| PPA | 96.8% | 92.3% | 100.0% |
| OA | 95.6% | 92.5% | 98.1% |

The estimates for NPA, PPA, and GA for the intra-site reproducibility endpoints are presented in Table 6:

TABLE 6
Intra-Site (Inter-Day) Reproducibility Analysis, ≥2 +
Intensity ≥ 50% Cut-off
nsNSCLC, Intra-Site, ≥50 Cutoff
Specimen Distribution Summary

| | Score | | |
|---|---|---|---|
| Definition | Range | # Blocks | % Blocks |
| Negative | <50 | 25/50 | 50.0% |
| Positive | ≥50 | 25/50 | 50.0% |
| Total Near Cutoff | 40-60 | 12/50 | 24.0% |

Comparisons to Consensus Summary

| | |
|---|---|
| Negative Consensus | Positive Consensus |

| Concordant | Discordant | Concordant | Discordant | |
|---|---|---|---|---|
| Negatives | Positives | Positives | Negatives | Total |
| 359 | 11 | 373 | 7 | 750 |

Agreement Summary

| Performance | Point | 95% Confidence Interval (Bootstrap) | |
|---|---|---|---|
| Criteria | Estimate | Lower-bound: 2.5% | Upper-bound: 97.5% |
| NPA | 97.0% | 94.9% | 98.9% |
| PPA | 98.2% | 96.4% | 99.5% |
| OA | 97.6% | 96.3% | 98.8% |

The lower bound of a two-sided 95% confidence interval for NPA, PPA, and OA were all greater than 85% for each reproducibility measure, thus the acceptance criteria were met for all analyses in Part A.

### Inter-Observer, Intra-Observer Reproducibility (Part B)/Summary of Experimental Design and Data Recording (Part B)

The objective of this study was to demonstrate that the Agilent scoring method and interpretation guideline for the CEACAM5 IHC 769 assay produce consistent results in normal observer-to-observer testing.

The data were analyzed to determine the concordance of the assay performance across the three sites based on the ≥2+ Intensity ≥50% cut-off. The same data were used for inter-observer (Table 7) and intra-observer analysis (Table 8).

### Inter-Observer Reproducibility Results

### NPA/PPA/OA

The estimate for NPA, PPA, and OA for the Inter-Observer reproducibility endpoints are presented in Table 7:

TABLE 7
Inter-Observer Reproducibility Analysis, ≥2 + Intensity ≥ 50% Cut-off
Table 7. Inter-Observer Reproducibility
Analysis, ≥2 + Intensity ≥ 50% Cut-off
nsNSCLC, Inter-Observer, ≥50 Cutoff
Specimen Distribution Summary

| Definition | Score Range | # Blocks | % Blocks |
|---|---|---|---|
| Negative | <50 | 34/66 | 51.5% |
| Positive | ≥50 | 32/66 | 48.5% |
| Total Near Cutoff | 40-60 | 11/66 | 16.7% |

### Intra-Observer Reproducibility Statistical Analysis

### NPA/PPA/OA

The estimates for NPA, PPA, and OA for the Intra-Observer reproducibility endpoints are presented in Table 8:

TABLE 8
Intra-Observer Reproducibility Analysis, ≥2 +
Intensity ≥50% Cut-off
nsNSCLC, Intra-Observer, ≥≥50 Cutoff
Specimen Distribution Summary

| Definition | Score Range | # Blocks | % Blocks |
|---|---|---|---|
| Negative | <50 | 34/66 | 51.5% |
| Positive | ≥50 | 32/66 | 48.5% |
| Total Near Cutoff | 40-60 | 11/66 | 16.7% |

Comparisons to Consensus Summary

| | |
|---|---|
| Negative Consensus | Positive Consensus |

| Concordant | Discordant | Concordant | Discordant | |
|---|---|---|---|---|
| Negatives | Positives | Positives | Negatives | Total |
| 293 | 1 | 295 | 5 | 594 |

Agreement Summary

| Performance | Point | 95% Confidence Interval (Bootstrap) | |
|---|---|---|---|
| Criteria | Estimate | Lower-bound: 2.5% | Upper-bound: 97.5% |
| NPA | 99.7% | 99.0% | 100.0% |
| PPA | 98.3% | 96.9% | 99.7% |
| OA | 99.0% | 98.1% | 99.7% |

The lower bound of a two-sided 95% confidence interval for NPA, PPA, and OA were all greater than 85% for each reproducibility measure, thus the acceptance criteria were met for all analyses in Part B.

### Summary Representation of the Diagnostic Estimates of Reproducibility Study

The results of the four study objectives presented above (Intra-site, Inter-site, Intra-observer and Inter-observer) with 95% confidence intervals are summarized in: FIG. 20: Summary of Results for the ≥2+ Intensity ≥50% Cut-off.

### Variance Component Analysis

The underlying continuous raw CEACAM5 scores in the reproducibility study were also evaluated based on an analysis of variance model (ANOVA). Values of mean, standard deviation (SD) and coefficient of variation (% CV) were calculated and data from both study Part A and Part B was analyzed and presented in Tables 9 and 10, respectively. It should be noted that no acceptance criteria were applied to these results. For some specimens, some or all variance components were not estimable due to lack of variability in scores (for example, all scores of 0).

TABLE 9
ANOVA Mixed Model on 50 Specimens from Study Part A: ANOVA Mixed Model
on 50 nsNSCLC Specimens Processed at Three Sites and on Five Days at each Site
nsNSCLC, ANOVA, Study Part A

| | | | | | | | | Total (Site + | |
|---|---|---|---|---|---|---|---|---|---|
| | # | Mean | Inter-Site | | Inter-Round | | Residual | Round + Residual) | |
| Block | Scores | Score | SD | CV | SD | CV | SD | SD | CV |
| 114672 | 15 | 3.70 | 0.00 | 0.00 | 0.00 | 0.00 | 3.23 | 3.23 | 0.87 |
| 116522 | 15 | 65.00 | 0.00 | 0.00 | 3.12 | 0.05 | 6.45 | 7.17 | 0.11 |
| 116580 | 15 | 93.00 | 0.00 | 0.00 | 0.00 | 0.00 | 4.14 | 4.14 | 0.04 |
| 117634 | 15 | 86.00 | 5.89 | 0.07 | 0.00 | 0.00 | 3.42 | 6.81 | 0.08 |
| 117637 | 15 | 76.67 | 6.42 | 0.08 | 2.14 | 0.03 | 3.93 | 7.83 | 0.10 |
| 117793 | 15 | 81.33 | 7.16 | 0.09 | 1.12 | 0.01 | 5.95 | 9.38 | 0.12 |
| 118398 | 15 | 0.00 | 0.00 | N/A | 0.00 | N/A | 0.00 | 0.00 | N/A |
| 123834 | 15 | 0.00 | 0.00 | N/A | 0.00 | N/A | 0.00 | 0.00 | N/A |
| 124116 | 15 | 36.00 | 2.12 | 0.06 | 0.00 | 0.00 | 4.74 | 5.20 | 0.14 |
| 124257 | 15 | 22.67 | 9.81 | 0.43 | 1.94 | 0.09 | 6.74 | 12.06 | 0.53 |
| 124455 | 15 | 0.00 | 0.00 | N/A | 0.00 | N/A | 0.00 | 0.00 | N/A |
| 125080 | 15 | 36.33 | 11.34 | 0.31 | 0.00 | 0.00 | 6.19 | 12.92 | 0.36 |
| 125102 | 15 | 72.33 | 4.40 | 0.06 | 0.00 | 0.00 | 5.00 | 6.66 | 0.09 |
| 125128 | 15 | 65.13 | 1.00 | 0.02 | 1.08 | 0.02 | 5.85 | 6.04 | 0.09 |
| 125144 | 15 | 3.03 | 2.94 | 0.97 | 0.45 | 0.15 | 4.73 | 5.58 | 1.84 |
| 125153 | 15 | 0.03 | 0.00 | 0.00 | 0.00 | 0.00 | 0.13 | 0.13 | 3.87 |
| 127344 | 15 | 0.93 | 0.67 | 0.72 | 0.00 | 0.00 | 0.77 | 1.03 | 1.10 |
| 127541 | 15 | 5.93 | 2.59 | 0.44 | 0.00 | 0.00 | 2.14 | 3.36 | 0.57 |
| 127697 | 15 | 60.33 | 11.15 | 0.18 | 0.00 | 0.00 | 6.71 | 13.01 | 0.22 |
| 128644 | 15 | 89.00 | 2.33 | 0.03 | 4.61 | 0.05 | 4.23 | 6.68 | 0.08 |
| 128673 | 15 | 36.00 | 2.94 | 0.08 | 0.00 | 0.00 | 12.32 | 12.66 | 0.35 |
| 128691 | 15 | 40.00 | 5.70 | 0.14 | 0.91 | 0.02 | 4.74 | 7.47 | 0.19 |
| 129255 | 15 | 62.33 | 6.22 | 0.10 | 1.44 | 0.02 | 6.16 | 8.87 | 0.14 |
| 129265 | 15 | 14.20 | 7.39 | 0.52 | 1.68 | 0.12 | 4.50 | 8.82 | 0.62 |
| 129309 | 15 | 62.67 | 3.70 | 0.06 | 0.00 | 0.00 | 4.28 | 5.66 | 0.09 |
| 129330 | 15 | 73.33 | 3.03 | 0.04 | 1.83 | 0.02 | 5.99 | 6.95 | 0.09 |
| 129341 | 15 | 1.53 | 0.58 | 0.38 | 0.92 | 0.60 | 1.11 | 1.55 | 1.01 |
| 129375 | 15 | 82.00 | 3.54 | 0.04 | 1.29 | 0.02 | 4.18 | 5.63 | 0.07 |
| 129376 | 15 | 23.00 | 6.80 | 0.30 | 3.48 | 0.15 | 8.59 | 11.49 | 0.50 |
| 130386 | 15 | 69.33 | 10.88 | 0.16 | 2.74 | 0.04 | 5.48 | 12.48 | 0.18 |
| 130390 | 15 | 92.67 | 3.81 | 0.04 | 0.00 | 0.00 | 3.76 | 5.35 | 0.06 |
| 130393 | 15 | 66.67 | 9.60 | 0.14 | 4.23 | 0.06 | 4.61 | 11.46 | 0.17 |
| 130490 | 15 | 68.00 | 5.16 | 0.08 | 2.42 | 0.04 | 6.83 | 8.90 | 0.13 |
| 130512 | 15 | 16.87 | 9.03 | 0.54 | 3.66 | 0.22 | 3.92 | 10.50 | 0.62 |
| 130523 | 15 | 74.33 | 5.21 | 0.07 | 0.00 | 0.00 | 3.98 | 6.56 | 0.09 |
| 130562 | 15 | 34.33 | 14.41 | 0.42 | 0.00 | 0.00 | 5.77 | 15.52 | 0.45 |
| 130572 | 15 | 3.53 | 1.63 | 0.46 | 1.58 | 0.45 | 1.71 | 2.85 | 0.81 |
| 132498 | 15 | 3.63 | 1.90 | 0.52 | 1.02 | 0.28 | 2.35 | 3.19 | 0.88 |
| 132500 | 15 | 52.67 | 10.08 | 0.19 | 3.16 | 0.06 | 6.58 | 12.45 | 0.24 |
| 134445 | 15 | 65.00 | 11.57 | 0.18 | 0.00 | 0.00 | 5.08 | 12.64 | 0.19 |
| 135209 | 15 | 91.00 | 1.58 | 0.02 | 2.42 | 0.03 | 4.74 | 5.55 | 0.06 |
| 135274 | 15 | 37.33 | 3.10 | 0.08 | 1.44 | 0.04 | 5.81 | 6.74 | 0.18 |
| 135275 | 15 | 78.00 | 8.54 | 0.11 | 1.71 | 0.02 | 5.52 | 10.31 | 0.13 |
| 137768 | 15 | 48.33 | 4.74 | 0.10 | 3.42 | 0.07 | 4.38 | 7.30 | 0.15 |
| 137820 | 15 | 39.67 | 10.63 | 0.27 | 2.14 | 0.05 | 4.13 | 11.60 | 0.29 |
| 137833 | 15 | 77.33 | 7.18 | 0.09 | 0.00 | 0.00 | 5.85 | 9.26 | 0.12 |
| 138799 | 15 | 59.00 | 1.63 | 0.03 | 0.02 | 0.00 | 8.17 | 8.33 | 0.14 |
| 138817 | 15 | 44.67 | 2.68 | 0.06 | 0.00 | 0.00 | 3.29 | 4.24 | 0.09 |
| 85623 | 15 | 38.67 | 7.94 | 0.21 | 0.00 | 0.00 | 5.63 | 9.73 | 0.25 |
| 85671 | 15 | 98.33 | 2.58 | 0.03 | 0.02 | 0.00 | 2.89 | 3.87 | 0.04 |

TABLE 10
ANOVA Mixed Model on 66 Specimens from Study Part B: ANOVA Mixed
Model on 66 nsNSCLC Specimens for Three Observers, each for Three Reads
nsNSCLC, ANOVA, Study Part B

| | | | | | | | | Total (Observer + | |
|---|---|---|---|---|---|---|---|---|---|
| | # | Mean | Inter-Observer | | Inter-Read | | Residual | Read + Residual) | |
| Block | Scores | Score | SD | CV | SD | CV | SD | SD | CV |
| 102970 | 9 | 31.67 | 0.00 | 0.00 | 0.00 | 0.00 | 7.91 | 7.91 | 0.25 |
| 114672 | 9 | 4.22 | 1.94 | 0.46 | 3.93 | 0.93 | 5.64 | 7.14 | 1.69 |
| 114675 | 9 | 100.00 | 0.00 | 0.00 | 0.00 | 0.00 | 0.00 | 0.00 | 0.00 |
| 116522 | 9 | 69.44 | 6.12 | 0.09 | 5.65 | 0.08 | 2.64 | 8.74 | 0.13 |
| 116580 | 9 | 92.22 | 6.38 | 0.07 | 0.00 | 0.00 | 3.73 | 7.39 | 0.08 |
| 117634 | 9 | 87.78 | 4.51 | 0.05 | 0.00 | 0.00 | 4.08 | 6.09 | 0.07 |
| 117637 | 9 | 86.67 | 2.55 | 0.03 | 0.00 | 0.00 | 3.73 | 4.51 | 0.05 |
| 117793 | 9 | 82.22 | 11.47 | 0.14 | 0.00 | 0.00 | 7.64 | 13.78 | 0.17 |
| 118398 | 9 | 0.00 | 0.00 | N/A | 0.00 | N/A | 0.00 | 0.00 | N/A |
| 123834 | 9 | 0.11 | 0.00 | 0.00 | 0.00 | 0.00 | 0.33 | 0.33 | 3.00 |
| 123848 | 9 | 11.44 | 3.82 | 0.33 | 0.00 | 0.00 | 4.64 | 6.02 | 0.53 |
| 124116 | 9 | 41.67 | 10.67 | 0.26 | 2.36 | 0.06 | 4.08 | 11.67 | 0.28 |
| 124117 | 9 | 91.67 | 0.96 | 0.01 | 0.00 | 0.00 | 2.36 | 2.55 | 0.03 |
| 124257 | 9 | 27.78 | 4.71 | 0.17 | 4.71 | 0.17 | 3.33 | 7.45 | 0.27 |
| 124455 | 9 | 0.11 | 0.00 | 0.00 | 0.00 | 0.00 | 0.33 | 0.33 | 3.00 |
| 125080 | 9 | 25.56 | 9.18 | 0.36 | 0.00 | 0.00 | 7.64 | 11.94 | 0.47 |
| 125102 | 9 | 80.00 | 9.57 | 0.12 | 0.00 | 0.00 | 7.07 | 11.90 | 0.15 |
| 125128 | 9 | 62.78 | 13.94 | 0.22 | 2.36 | 0.04 | 4.41 | 14.81 | 0.24 |
| 125144 | 9 | 2.11 | 2.40 | 1.14 | 0.03 | 0.01 | 2.60 | 3.54 | 1.68 |
| 125153 | 9 | 0.00 | 0.00 | N/A | 0.00 | N/A | 0.00 | 0.00 | N/A |
| 127344 | 9 | 0.78 | 0.00 | 0.00 | 0.19 | 0.25 | 1.29 | 1.31 | 1.68 |
| 127533 | 9 | 63.89 | 9.86 | 0.15 | 0.00 | 0.00 | 6.67 | 11.90 | 0.19 |
| 127541 | 9 | 5.67 | 3.32 | 0.59 | 0.00 | 0.00 | 2.45 | 4.12 | 0.73 |
| 127682 | 9 | 20.00 | 4.51 | 0.23 | 0.00 | 0.00 | 6.87 | 8.22 | 0.41 |
| 127697 | 9 | 74.44 | 0.00 | 0.00 | 0.00 | 0.00 | 9.82 | 9.82 | 0.13 |
| 128644 | 9 | 85.56 | 4.71 | 0.06 | 8.16 | 0.10 | 3.33 | 10.00 | 0.12 |
| 128673 | 9 | 30.00 | 3.04 | 0.10 | 0.00 | 0.00 | 5.53 | 6.31 | 0.21 |
| 128691 | 9 | 41.11 | 6.12 | 0.15 | 2.04 | 0.05 | 4.86 | 8.08 | 0.20 |
| 129255 | 9 | 64.44 | 1.67 | 0.03 | 3.33 | 0.05 | 3.33 | 5.00 | 0.08 |
| 129265 | 9 | 10.78 | 6.17 | 0.57 | 1.75 | 0.16 | 4.43 | 7.79 | 0.72 |
| 129290 | 9 | 78.33 | 7.52 | 0.10 | 0.00 | 0.00 | 9.43 | 12.06 | 0.15 |
| 129309 | 9 | 66.11 | 1.67 | 0.03 | 1.67 | 0.03 | 6.67 | 7.07 | 0.11 |
| 129329 | 9 | 6.00 | 4.56 | 0.76 | 0.00 | 0.00 | 3.73 | 5.89 | 0.98 |
| 129330 | 9 | 66.67 | 11.78 | 0.18 | 2.36 | 0.04 | 4.08 | 12.69 | 0.19 |
| 129341 | 9 | 1.17 | 0.00 | 0.00 | 0.00 | 0.00 | 1.32 | 1.32 | 1.13 |
| 129376 | 9 | 28.33 | 1.67 | 0.06 | 0.00 | 0.00 | 4.08 | 4.41 | 0.16 |
| 130386 | 9 | 73.33 | 5.89 | 0.08 | 1.18 | 0.02 | 7.36 | 9.50 | 0.13 |
| 130390 | 9 | 91.11 | 6.24 | 0.07 | 0.04 | 0.00 | 4.41 | 7.64 | 0.08 |
| 130393 | 9 | 79.44 | 9.77 | 0.12 | 0.00 | 0.00 | 5.00 | 10.97 | 0.14 |
| 130490 | 9 | 72.22 | 12.06 | 0.17 | 0.00 | 0.00 | 8.16 | 14.56 | 0.20 |
| 130512 | 9 | 7.33 | 3.04 | 0.41 | 0.00 | 0.00 | 2.13 | 3.72 | 0.51 |
| 130523 | 9 | 80.00 | 7.07 | 0.09 | 0.00 | 0.00 | 5.00 | 8.66 | 0.11 |
| 130547 | 9 | 79.44 | 6.56 | 0.08 | 4.56 | 0.06 | 11.12 | 13.69 | 0.17 |
| 130562 | 9 | 30.56 | 0.00 | 0.00 | 0.00 | 0.00 | 6.82 | 6.82 | 0.22 |
| 130572 | 9 | 3.11 | 0.00 | 0.00 | 0.00 | 0.00 | 3.22 | 3.22 | 1.03 |
| 132498 | 9 | 2.44 | 3.52 | 1.44 | 0.00 | 0.00 | 1.80 | 3.95 | 1.62 |
| 132500 | 9 | 53.89 | 0.96 | 0.02 | 0.00 | 0.00 | 4.08 | 4.19 | 0.08 |
| 134445 | 9 | 71.11 | 9.28 | 0.13 | 3.33 | 0.05 | 4.41 | 10.80 | 0.15 |
| 135209 | 9 | 94.44 | 0.00 | 0.00 | 2.36 | 0.02 | 6.01 | 6.45 | 0.07 |

### DISCUSSION/CONCLUSIONS

The data in this Example provide an estimate of the reproducibility parameters of CEACAM5 IHC 769 at the ≥2+ Intensity ≥50% cut-off within the context of the evaluation of nsNSCLC cases. The study met all acceptance criteria. The calculated lower limit of the two-sided 95% confidence intervals was greater than or equal to 85% in all endpoints of the ≥2+ Intensity ≥50% cut-off analysis.

A number of embodiments of the invention have been described. Nevertheless, it can be understood that various modifications may be made without departing from the spirit and scope of the invention. Accordingly, other embodiments are within the scope of the following claims.

### Alternative Embodiments

In alternative embodiments, provided are immunohistochemistry (IHC) methods for determining and scoring the extent of cellular membrane expression of carcinoembryonic antigen-related cell adhesion molecule 5, or CEACAM5 (also known as CD66e (Cluster of Differentiation 66e)) in a tissue sample, comprising:
staining a tissue sample with an antibody which specifically binds to CEACAM5;
determining a total number of viable tumor or cancer cells having CEACAM5 cellular membrane staining, and determining a total number of staining and non-staining viable tumor or cancer cells in at least a portion of the tissue sample,
wherein a tumor or cancer cell is counted as positively stained with
anti-CEACAM5 antibody if there is CEACAM5 cellular membrane staining at any intensity above a defined threshold; and
determining a Tumor Intensity Proportion Score,
wherein the Tumor Intensity Proportion Score is the number of CEACAM5 staining viable tumor or cancer cells found in the tissue sample divided by the total number of staining and non-staining viable tumor or cancer cells, multiplied by 100.

In alternative embodiments of IHC methods as provided herein:
the CEACAM5 cellular membrane staining comprises whole membrane staining, discontinuous membrane staining, partial membrane staining, complete (circumferential) membrane staining, punctate membrane staining, linear membrane staining, luminal membrane staining, apical membrane staining, basal membrane staining, lateral membrane staining, basolateral membrane staining, high cytoplasmic staining at a 2+ or greater positive staining intensity in the signet ring subtype, high cytoplasmic staining at a 3+ positive staining intensity when cells within a 10× field of view have unambiguous membrane staining, or a combination thereof;
the total number of viable tumor or cancer cells comprises all viable tumor or cancer cells in the specimen, including luminal cells, glandular luminal cells, intraluminal cells, multiple layers of cells, cells having signet ring morphology, high cytoplasmic staining cells, cells with intracytoplasmic lumina, or a combination thereof;
the total number of viable tumor or cancer cells counted as positively stained comprises all glandular luminal cells having a cellular membrane associated with the positively staining lumen;
the tissue sample comprises at least about 100 viable tumor or cancer cells;
a tumor or cancer cell counted as positively stained excludes tumor or cancer cells having exclusively cytoplasmic staining (with exceptions), staining of normal or non-neoplastic structures, staining nonviable tumor or cancer cells, necrotic cells, cellular debris, stromal staining, or edge artifact staining on a periphery of the tissue sample;
cytoplasmic staining comprises cytoplasmic staining at a 2+ or lesser positive staining intensity; or, the defined threshold comprises a 2+ or greater positive staining intensity; or, the defined threshold comprises a 2+ positive or a 3+ positive staining intensity evaluated at a magnification of from about 4× to about 10×; or further comprising confirming the positive staining intensity at a magnification of from about 20× to about 40×;
the Tumor Intensity Proportion Score (TIPS) comprises the number of CEACAM5 viable tumor or cancer cells staining at a 2+ or greater positive staining intensity divided by the total number of staining and non-staining viable tumor or cancer cells, multiplied by 100; or, a TIPS of about 40% or greater indicates a diagnostic status of the tissue sample; or, the TIPS is about 50% or greater, about 60% or greater, about 80% or greater, or about 90% or greater;
a TIPS of about 5% or greater indicates a diagnostic status of the tissue sample; or, a TIPS of about 5% or greater comprises the number of CEACAM5 viable tumor or cancer cells staining at a 2+ or greater positive staining intensity; or a TIPS of about 10% or greater comprises the number of CEACAM5 viable tumor or cancer cells staining at a 2+ or greater positive staining intensity;
a section or portion of the tissue sample is prepared on a slide, a microscope slide, or equivalent, and the section or portion of the tissue sample is stained on the slide;
the antibody comprises a monoclonal mouse anti-CEACAM5 antibody or a monoclonal rabbit anti-CEACAM5 antibody; or, the monoclonal mouse anti-CEACAM5 antibody comprises monoclonal mouse anti-CEACAM5 clone 769, or an antibody having a substantially similar affinity for CEACAM5 as a clone 769 antibody;
the tissue sample comprises a formalin-fixed, paraffin-embedded (FFPE) specimen; or, the FFPE specimen comprises a cancer specimen stained on an automated IHC platform;
the section of the tissue sample is prepared by a protocol comprising fixation in about 10% neutral buffered formalin for a time period of about 6 hours to about 72 hours;
the tumor or cancer is a non-squamous non-small cell lung cancer (nsNSCLC), a non-small cell lung cancer (NSCLC), an adenocarcinoma, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a stomach cancer, a colorectal cancer, a lung cancer, a colon cancer, a breast cancer, an ovarian cancer, an endometrial cancer, a cervical cancer, a prostate cancer, or a urothelial cancer;
the tissue sample is derived from a surgical resection or biopsy, needle biopsy sample, a fine-needle aspirate, a cytology specimen, or a bone decalcification; and/or
a positive staining is determined using a bright-field light microscope, a microscope objective, a computer monitor and imaging software, or a combination thereof, and optionally, the imaging software comprises whole slide imaging software.

1. An immunohistochemistry (IHC) method for determining and scoring the extent of cellular membrane expression of carcinoembryonic antigen-related cell adhesion molecule 5 (CEACAM5) in a tissue sample, comprising:
(a) staining a tissue sample with an antibody which specifically binds to CEACAM5;
(b) determining a total number of viable tumor or cancer cells having CEACAM5 cellular membrane staining, and determining a total number of staining and non-staining viable tumor or cancer cells in at least a portion of the tissue sample,
wherein a tumor or cancer cell is counted as positively stained with anti-CEACAM5 antibody if there is CEACAM5 cellular membrane staining at any intensity above a defined threshold; and
(c) determining a Tumor Intensity Proportion Score,
wherein the Tumor Intensity Proportion Score is the number of CEACAM5 staining viable tumor or cancer cells found in the tissue sample divided by the total number of staining and non-staining viable tumor or cancer cells, multiplied by 100.

2. The method of claim 1, wherein the CEACAM5 cellular membrane staining comprises whole membrane staining, discontinuous membrane staining, partial membrane staining, complete membrane staining, punctate membrane staining, linear membrane staining, luminal membrane staining, apical membrane staining, basal membrane staining, lateral membrane staining, basolateral membrane staining, high cytoplasmic staining at a 2+ or greater positive staining intensity, high cytoplasmic staining at a 3+ positive staining intensity, or a combination thereof.

3. The method of claim 1, wherein the total number of viable tumor or cancer cells comprises luminal cells, glandular luminal cells, intraluminal cells, multiple layers of cells, cells having signet ring morphology, high cytoplasmic staining cells, cells with intracytoplasmic lumina, or a combination thereof.

4. The method of claim 1, wherein the total number of viable tumor or cancer cells counted as positively stained comprises all glandular luminal cells having a cellular membrane associated with the positively staining lumen.

5. The method of claim 1, wherein the tissue sample comprises at least about 100 cells.

6. The method of claim 1, wherein a tumor or cancer cell counted as positively stained excludes tumor or cancer cells having cytoplasmic staining, staining of normal or non-neoplastic structures, staining nonviable tumor or cancer cells, necrotic cells, cellular debris, stromal staining, or edge artifact staining on a periphery of the tissue sample, and optionally the cytoplasmic staining comprises cytoplasmic staining at a 2+ or lesser positive staining intensity.

7. (canceled)

8. The method of claim 1, wherein the defined threshold comprises a 2+ or greater positive staining intensity,
and optionally the defined threshold comprises a 2+ positive or a 3+ positive staining intensity evaluated at a magnification of from about 4× to about 10×; or further comprising confirming the positive staining intensity at a magnification of from about 20× to about 40×,
and optionally the Tumor Intensity Proportion Score comprises the number of CEACAM5 viable tumor or cancer cells staining at a 2+ or greater positive staining intensity divided by the total number of staining and non-staining viable tumor or cancer cells, multiplied by 100.

9-10. (canceled)

11. The method of claim 1, wherein a Tumor Intensity Proportion Score of about 40% or greater indicates a diagnostic status of the tissue sample,
and optionally the Tumor Intensity Proportion Score is about 50% or greater, about 60% or greater, about 80% or greater, or about 90% or greater,
and optionally the Tumor Intensity Proportion Score of about 5% or greater indicates a diagnostic status of the tissue sample,
and optionally the Tumor Intensity Proportion Score of about 5% or greater comprises the number of CEACAM5 viable tumor or cancer cells staining at a 2+ or greater positive staining intensity; or a Tumor Intensity Proportion Score of about 10% or greater comprises the number of CEACAM5 viable tumor or cancer cells staining at a 2+ or greater positive staining intensity.

12-14. (canceled)

15. The method of claim 1, wherein a section or portion of the tissue sample is prepared on a slide, a microscope slide, or equivalent, and the section or portion of the tissue sample is stained on the slide.

16. The method of claim 1, wherein the antibody comprises a monoclonal mouse anti-CEACAM5 antibody or a monoclonal rabbit anti-CEACAM5 antibody,
and optionally the monoclonal mouse anti-CEACAM5 antibody comprises monoclonal mouse anti-CEACAM5 clone 769, or an antibody having a substantially similar affinity for CEACAM5 as a clone 769 antibody.

17. (canceled)

18. The method of claim 1, wherein the tissue sample comprises a formalin-fixed, paraffin-embedded (FFPE) specimen,
and optionally, the FFPE specimen comprises a cancer specimen stained on an automated IHC platform.

19. (canceled)

20. The method of claim 18, wherein the section of the tissue sample is prepared by a protocol comprising fixation in about 10% neutral buffered formalin for a time period of about 6 hours to about 72 hours.

21. The method of claim 1, wherein the tumor or cancer is a non-squamous non-small cell lung cancer (nsNSCLC), a non-small cell lung cancer (NSCLC), an adenocarcinoma, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a stomach cancer, a colorectal cancer, a lung cancer, a colon cancer, a breast cancer, an ovarian cancer, an endometrial cancer, a cervical cancer, a prostate cancer, or a urothelial cancer.

22. The method of claim 1, wherein the tissue sample is derived from a needle biopsy sample, a fine-needle aspirate, a cytology specimen, or a bone decalcification.

23. The method of claim 1, wherein positive staining is determined using a bright-field light microscope, a microscope objective, a computer monitor and imaging software, or a combination thereof,
and optionally the imaging software comprises whole slide imaging software.

24. (canceled)

25. A method for diagnosing a tumor or a cancer by determining if a tissue sample is positive for expression of carcinoembryonic antigen-related cell adhesion molecule 5 (CEACAM5), comprising: determining a CEACAM5 diagnostic status in a tissue sample by a method of claim 1,
wherein a Tumor Intensity Proportion Score of about 50% or greater of tumor cancer cells having CEACAM5 cellular membrane staining at an intensity of 2+ or greater is diagnostically positive,
and optionally the tumor or cancer is a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer.

26. (canceled)

27. A method for treating or ameliorating a tumor or a cancer in a patient, comprising determining and scoring the amount of CEACAM5 in a tissue sample from the patient using a method of claim 1, wherein if the tissue sample is determined or scored to have a high or a diagnostically positive CEACAM5 score, the patient is treated with a cancer therapeutic to which the patient is likely to respond favorably,
and optionally the tumor or cancer is a non-squamous non-small cell lung cancer (nsNSCLC), a non-small cell lung cancer (NSCLC), an adenocarcinoma, a synovial sarcoma, a myxoid/round cell liposarcoma, a head and neck cancer, a melanoma an esophageal cancer, a gastric cancer, a colorectal cancer, a lung cancer, a colon cancer, a breast cancer, an ovarian cancer, an endometrial cancer, a cervical cancer, a prostate cancer, or a urothelial cancer,
and optionally the cancer therapeutic comprises administration to the patient an anti-cancer drug or an anti-cancer therapy,
and optionally the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (CAR) T-cell therapy.

28-30. (canceled)

31. A kit comprising an antibody which specifically binds to CEACAM5 and CEACAM5 scoring guidelines as set forth in claim 1.

32. A method for assessing the extent of CEACAM5 expression comprising: contacting a sample or a portion thereof comprising cancer or tumor cells from an individual with an antibody or a portion thereof which specifically binds to CEACAM5; and determining a Tumor Intensity Proportion Score by dividing the number of CEACAM5 staining viable tumor or cancer cells in the sample or portion thereof specifically bound by the antibody with the total number of staining and non-staining viable cancer or tumor cells and multiplying the result by 100, thereby obtaining the Tumor Intensity Proportion Score.
